(19) United States
(12) Patent Application Publication
(10) Pub. No.: US 2024/0423153 A1
(43) Pub. Date: Dec. 26, 2024

(54) METHODS AND COMPOSITIONS FOR PRODUCING CLONAL, NON-REDUCED, NON-RECOMBINED GAMETES

(71) Applicants: PIONEER HI-BRED INTERNATIONAL, INC., JOHNSTON, IA (US); CORTEVA AGRISCIENCE LLC, INDIANAPOLIS, IN (US)

(72) Inventors: MARC C ALBERTSEN, GRIMES, IA (US); TIM WAYNE FOX, DES MOINES, IA (US); MARISSA SIMON, GRIMES, IA (US); MARK E WILLIAMS, JOHNSTON, IA (US)

(73) Assignees: PIONEER HI-BRED INTERNATIONAL, INC., JOHNSTON, IA (US); CORTEVA AGRISCIENCE LLC, INDIANAPOLIS, IN (US)

(21) Appl. No.: 18/830,940

(22) Filed: Sep. 11, 2024

Related U.S. Application Data
(63) Continuation of application No. 16/085,838, filed on Sep. 17, 2018, now Pat. No. 12,133,495.
(60) Provisional application No. 62/310,006, filed on Mar. 18, 2016.

(51) Int. Cl. A01H 6/46; C07K 14/415; C12N 15/82

(52) U.S. Cl. CPC A01H 6/4684; C07K 14/415; C12N 15/8287

(57) ABSTRACT

Methods and compositions useful for avoiding plant meiosis and clonal reproduction through seed are provided herein. The present disclosure provides polynucleotides and related polypeptides of Spo11, Rec8, OSD1-1A, and OSD1-3A and methods and compositions for suppressing their expression level or activity.

### CROSS REFERENCE

This patent application which claims the benefit of and priority to U.S. Provisional Application No. 62/310,006, filed Mar. 18, 2016, which is incorporated herein by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named “7113WOPCT_SequenceListing” created on Mar. 9, 2017, and having a size of 69 kilobytes and is filed concurrently with the specification. The sequence listing contained in this ASCII formatted document is part of the specification and is herein incorporated by reference in its entirety.

### FIELD OF THE INVENTION

The field relates to apomixis and molecular biology.

### BACKGROUND

Sexual reproduction increases genetic diversity and is based on meiosis and fertilization. Meiosis has two meiotic germ cell divisions, meiosis I and meiosis II. During the first division (meiosis I), paternal and maternal chromosomes are able to cross-over and exchange genes before the pairs of chromosomes are separated into two haploid cells. Each resulting haploid cell contains only half the number of chromosomes and two chromatid pairs. The second meiotic division (meiosis II) segregates the sister chromatids and results in the formation of four haploid gametes. A gamete from a male and female parent can unite to form a genetically unique diploid zygote.

In contrast, asexual reproduction does not involve the union of gametes. In some examples, asexual reproduction in plants can be achieved by avoiding meiosis. The avoidance of meiosis could allow for a parent plant to produce gametes that are genetically identical to itself.

### SUMMARY

One embodiment of the current disclosure is a method of obtaining a maize plant that produces clonal, non-reduced, non-recombined gametes by suppressing in the maize plant the activity of an endogenous Spo11 polynucleotide or polypeptide, an endogenous Rec8 (aka Afd1 in maize) polynucleotide or polypeptide, an endogenous OSD1-1A polynucleotide or polypeptide, and an endogenous OSD1-3A polynucleotide or polypeptide. Accordingly, one embodiment is a maize plant that produces clonal, non-reduced, non-recombined gametes, where the maize plant comprises suppressed activity of an endogenous Spo11 polynucleotide or polypeptide, an endogenous Rec8 polynucleotide or polypeptide, an endogenous OSD1-1A polynucleotide or polypeptide, and an endogenous OSD1-3A polynucleotide or polypeptide. In some embodiments, the Spo11 polynucleotide is selected from the group consisting of: a polynucleotide that encodes the polypeptide of SEQ ID NO:16; a polynucleotide comprising the sequence set forth in SEQ ID NO:13, 14, 15, or 19; and a polynucleotide having at least 80% sequence identity to SEQ ID NO: 13, 14, 15, or 19 and the Spo11 polypeptide is selected from the group consisting of: a polypeptide comprising SEQ ID NO: 16; a polypeptide that is at least 80% identical to the amino acid sequence of SEQ ID NO:16; a polypeptide that is encoded by a nucleic acid molecule comprising a nucleotide sequence that is at least 80% identical to the sequence set forth in SEQ ID NO:13, 14 or 15. In some embodiments, the Rec8 polynucleotide is selected from the group consisting of: a polynucleotide that encodes the polypeptide of SEQ ID NO:12, a polynucleotide comprising the sequence set forth in SEQ ID NO:9, 10, 11 or 20; and a polynucleotide having at least 80% sequence identity to SEQ ID NO:9, 10, 11 or 20; and the Rec8 polypeptide is selected from the group consisting of: a polypeptide comprising SEQ ID NO: 12; a polypeptide that is at least 80% identical to the amino acid sequence of SEQ ID NO:12; a polypeptide that is encoded by a nucleic acid molecule comprising a nucleotide sequence that is at least 80% identical to the sequence set forth in SEQ ID NO:9, 10 or 11. In some embodiments, the endogenous OSD1-1A polynucleotide or polypeptide is selected from the group consisting of: a polynucleotide that encodes the polypeptide of SEQ ID NO:4; a polynucleotide comprising the sequence set forth in SEQ ID NO:1, 2, 3 or 21; and a polynucleotide having at least 80% sequence identity to the sequence set forth in SEQ ID NO: 1, 2, 3, or 21; and the OSD1-1A polypeptide is selected from the group consisting of: a polypeptide comprising SEQ ID NO:4; a polypeptide that is at least 80% identical to the amino acid sequence of SEQ ID NO:4; a polypeptide that is encoded by a nucleic acid molecule comprising a nucleotide sequence that is at least 80% identical to the sequence set forth in SEQ ID NO:1, 2 or 3. In some embodiments, the endogenous OSD1-3A polynucleotide is selected from the group consisting of: a polynucleotide that encodes the polypeptide of SEQ ID NO:8; a polynucleotide comprising the sequence set forth in SEQ ID NO:5, 6, 7, or 22; and a polynucleotide having at least 80% sequence identity to the sequence set forth in SEQ ID NO:5, 6, 7, or 22, and the OSD1-3A polypeptide is selected from the group consisting of: a polypeptide comprising SEQ ID NO:8; a polypeptide that is at least 80% identical to the amino acid sequence of SEQ ID NO:8; a polypeptide that is encoded by a nucleic acid molecule comprising a nucleotide sequence that is at least 80% identical to the sequence set forth in SEQ ID NO: 5, 6, or 7.

In certain embodiments, the activity of the endogenous Spo11 polynucleotide or polypeptide, Rec8 polynucleotide or polypeptide, OSD1-1A polynucleotide or polypeptide, and/or OSD1-3A polynucleotide or polypeptide, or combinations thereof is suppressed using genome editing. In certain embodiments the suppression is a knock-out of the gene. In some approaches, the suppression results from a nucleotide modification of one or more the endogenous Spo11, Rec8, OSD1-1A or OSD1-3A polynucleotide sequences. In some embodiments, the nucleotide modification is a deletion, addition, or substitution of one or more nucleotides. In certain embodiments, the suppression results from an amino modification of one or more the endogenous Spo11, Rec8, OSD1-1A or OSD1-3A polypeptide sequences. In some embodiments, the amino acid modification is a deletion, addition, or substitution of one or more amino acids. In certain embodiments, the maize plant is a hybrid. In some embodiments, the activity of the endogenous Spo11 polynucleotide or polypeptide, Rec8 polynucleotide or polypeptide, OSD1-1A polynucleotide or polypeptide, OSD1-3A polynucleotide or polypeptide, or combinations thereof is suppressed using RNA-based silencing approaches, for example, antisense, microRNA, RNAi, or hairpin molecule, described elsewhere herein. In certain embodiments, the maize plant is a hybrid. In certain embodiments, the maize plant is an inbred. In certain embodiments, the maize plant is a female or male parent. Gametes obtained from this plant may be clonal, non-reduced, non-recombined gametes.

One embodiment of the disclosure includes a method of obtaining a maize plant that produces clonal, non-reduced, non-recombined gametes by crossing maize plants that are heterozygous or homozygous for the suppressed endogenous Spo11, Rec8, OSD1-1A and OSD1-3A activity with one another until a maize plant is obtained that is homozygous for the suppressed endogenous Spo11, Rec8, OSD1-1A and OSD1-3A activity. In certain embodiments, the maize plant is a hybrid. In certain embodiments, the maize plant is an inbred. In certain embodiments, the maize plant is a female or male parent. Gametes obtained from this plant may be clonal, non-reduced, non-recombined gametes.

In certain embodiments, the disclosure includes a method for obtaining a maize plant with a modified endogenous Spo11 polynucleotide sequence. In certain aspects, the modified endogenous Spo11 disrupts the homologous pairing and subsequent recombination of chromosomes during meiosis in plants. In certain aspects, the activity of the endogenous Spo11 polynucleotide or polypeptide is suppressed using genome editing approaches, for example, by genetically modifying the endogenous Spo11. In some approaches, the suppression results from a nucleotide modification of the endogenous maize Spo11 polynucleotide sequences. In some embodiments, the nucleotide modification is a deletion, addition, or substitution of one or more nucleotides. In certain embodiments, the suppression results from an amino acid modification of the endogenous Spo11 polypeptide sequence. In some embodiments, the amino acid modification is a deletion, addition, or substitution of one or more amino acids. In certain aspects, the activity of the endogenous Spo11 polynucleotide or polypeptide is suppressed using RNA-based silencing approaches, for example, antisense, microRNA, RNAi, or hairpin molecule, described elsewhere herein. In certain embodiments, the suppression of Spo11 is a knock-out of the gene.

In certain embodiments, the disclosure includes a method for obtaining a maize plant with a modified endogenous Rec8 polynucleotide sequence. In certain aspects, the modified endogenous Rec8 disrupts the orientation of kinetochores and subsequent random distribution of chromatids during meiosis II in a plant cell. In certain aspects, the activity of the endogenous Rec8 polynucleotide or polypeptide is suppressed using genome editing approaches, for example, by genetically modifying the endogenous Rec8. In some approaches, the suppression results from a nucleotide modification of the endogenous maize Rec8 polynucleotide sequences. In some embodiments, the nucleotide modification is a deletion, addition, or substitution of one or more nucleotides. In certain embodiments, the suppression results from an amino acid modification of the endogenous Rec8 polypeptide sequence. In some embodiments, the amino acid modification is a deletion, addition, or substitution of one or more amino acids. In certain aspects, the activity of the endogenous Rec8 polynucleotide or polypeptide is suppressed using RNA-based silencing approaches, for example, antisense, microRNA, RNAi, or hairpin molecule, described elsewhere herein. In certain embodiments, the suppression of Rec8 is a knock-out of the gene.

In certain embodiments, the disclosure includes a method for obtaining a maize plant with a modified endogenous OSD1-1A polynucleotide sequence. In certain aspects, the modified endogenous OSD1-1A and modified endogenous OSD1-3A together disrupt the progression of meiosis II division in a plant cell and produce non-reduced gametes. In certain aspects, the activity of the endogenous OSD1-1A polynucleotide or polypeptide is suppressed using genome editing approaches, for example, by genetically modifying the endogenous Rec8. In some approaches, the suppression results from a nucleotide modification of one or more the endogenous maize OSD1-1A polynucleotide sequences. In some embodiments, the nucleotide modification is a deletion, addition, or substitution of one or more nucleotides. In certain embodiments, the suppression results from an amino acid modification of the endogenous OSD1-1A polypeptide sequence. In some embodiments, the amino acid modification is a deletion, addition, or substitution of one or more amino acids. In certain aspects, the activity of the endogenous OSD1-1A polynucleotide or polypeptide is suppressed using RNA-based silencing approaches, for example, antisense, microRNA, RNAi, or hairpin molecule, described elsewhere herein. In certain embodiments, the suppression of OSD1-1A is a knock-out of the gene.

In certain embodiments, the disclosure includes a method for obtaining a maize plant with a modified endogenous OSD1-3A polynucleotide sequence. In certain aspects, the modified endogenous OSD1-1A and the modified endogenous OSD1-3A together disrupt the progression of meiosis II division in a plant cell and produce non-reduced gametes. In certain aspects, the activity of the endogenous OSD1-3A polynucleotide or polypeptide is suppressed using genome editing approaches, for example, by genetically modifying the endogenous Rec8. In some approaches, the suppression results from a nucleotide modification of one or more the endogenous maize OSD1-3A polynucleotide sequences. In some embodiments, the nucleotide modification is a deletion, addition, or substitution of one or more nucleotides. In certain embodiments, the suppression results from an amino acid modification of the endogenous OSD1-3A polypeptide sequence. In some embodiments, the amino acid modification is a deletion, addition, or substitution of one or more amino acids. In certain aspects, the activity of the endogenous OSD1-3A polynucleotide or polypeptide is suppressed using RNA-based silencing approaches, for example, antisense, microRNA, RNAi, or hairpin molecule, described elsewhere herein. In certain embodiments, the suppression of OSD1-3A is a knock-out of the gene.

### BRIEF DESCRIPTION OF THE SEQUENCE LISTING

The disclosure can be more fully understood from the following detailed description and the accompanying Sequence Listing which form a part of this application.

Table 1 presents SEQ ID NOs for the Spo11, Rec8, OSD1-1A, OSD1-3A polynucleotide and polypeptide sequences from Zea mays and maize OSD1-1A and OSD1-3A amiRNA target sequences. It is understood, as those skilled in the art will appreciate, that the disclosure encompasses more than these specific exemplary sequences.

TABLE 1

| Plant | Sequence Name | SEQ ID NO: |
|---|---|---|
| maize | OSD1-1A genomic DNA | 1 |
| maize | OSD1-1A cDNA | 2 |
| maize | OSD1-1A CDS (coding) | 3 |
| maize | OSD1-1A amino acid | 4 |
| maize | OSD1-3A genomic DNA | 5 |
| maize | OSD1-3A cDNA | 6 |
| maize | OSD1-3A CDS (coding) | 7 |
| maize | OSD1-3A amino acid | 8 |
| maize | Rec8 genomic DNA | 9 |
| maize | Rec8 cDNA | 10 |
| maize | Rec8 CDS (coding) | 11 |
| maize | Rec8 amino acid | 12 |
| maize | Spo11 genomic DNA | 13 |
| maize | Spo11 cDNA | 14 |
| maize | Spo11 CDS (coding) | 15 |
| maize | Spo11 amino acid | 16 |
| maize | OSD1-1A amiRNA target sequence | 17 |
| maize | OSD1-3A amiRNA target sequence | 18 |
| maize | Spo11 upstream sequence | 19 |
| maize | Rec8 upstream sequence | 20 |
| maize | OSD1-1A upstream sequence | 21 |
| maize | OSD1-3A upstream sequence | 22 |

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise.

In describing the present invention, the following terms will be employed, and are intended to be defined as indicated below.

The terms “altered target site”, “altered target sequence”, “modified target site”, and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii). Also, multiple mutations could be used in combination.

“Antisense inhibition” generally refers to the production of antisense RNA transcripts capable of suppressing the expression of the target gene or gene product. “Antisense RNA” generally refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

The terms “artificial target site” and “artificial target sequence” are used interchangeably herein and refer to a target sequence that has been introduced into the genome of a cell or organism. Such an artificial target sequence can be identical in sequence to an endogenous or native target sequence in the genome of a cell but be located in a different position (i.e., a non-endogenous or non-native position) in the genome of a cell or organism.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Cosuppression” generally refers to the production of sense RNA transcripts capable of suppressing the expression of the target gene or gene product. “Sense” RNA generally refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

“Developmentally regulated promoter” generally refers to a promoter whose activity is determined by developmental events.

The terms “dicot” and “dicotyledonous plant” are used interchangeably herein. A dicot of the current disclosure includes the following families: Brassicaceae, Leguminosae, and Solanaceae.

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

The terms “full complement” and “full-length complement” are used interchangeably herein, and refer to a complement of a given nucleotide sequence, wherein the complement and the nucleotide sequence consist of the same number of nucleotides and are 100% complementary.

“Gamete” refers to a reproductive cell having the 1n set (haploid number) of chromosomes that can fuse with another gamete of the opposite sex during fertilization in organisms undergoing sexual reproduction. As used herein, a gamete in organisms undergoing asexual reproduction refers to a cell having a 2n number (an unreduced number) of chromosomes.

“Genome” as it applies to plant cells encompasses not only chromosomal DNA found within the nucleus, but organelle DNA found within subcellular components (e.g., mitochondrial, plastid) of the cell.

“Heterologous” with respect to sequence means a sequence that originates from a foreign species, or, if from the same species, is substantially modified from its native form in composition and/or genomic locus by deliberate human intervention.

The term “introduced” in the context of inserting a nucleic acid into a cell, means “transfection” or “transformation” or “transduction” and includes reference to the incorporation of a nucleic acid or nucleic acid fragment into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. The term also embraces recombinant polynucleotides and chemically synthesized polynucleotides.

The terms “miRNA-star sequence” and “miRNA* sequence” are used interchangeably herein and they refer to a sequence in the miRNA precursor that is highly complementary to the miRNA sequence. The miRNA and miRNA* sequences form part of the stem region of the miRNA precursor hairpin structure.

The terms “monocot” and “monocotyledonous plant” are used interchangeably herein. A monocot of the current disclosure includes the Gramineae.

As used herein, “nucleic acid” includes reference to a deoxyribonucleotide or ribonucleotide polymer in either single- or double-stranded form.

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. In certain embodiments, sequence identity may be based on the Clustal V or Clustal W method of alignment. The term “about” when used herein in context with percent sequence identity means +/−1.0%.

“Phenotype” means the detectable characteristics of a cell or organism.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Sorghum, Avena, Hordeum, Secale, Allium and Triticum. A particularly preferred plant is Zea mays.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

“Polypeptide”, “peptide”, “amino acid sequence” and “protein” are used interchangeably herein to refer to a polymer of amino acid residues.

“Promoter functional in a plant” is a promoter capable of controlling transcription in plant cells whether or not its origin is from a plant cell.

As used herein “promoter” includes reference to a region of DNA upstream from the start of transcription and involved in recognition and binding of RNA polymerase and other proteins to initiate transcription.

A “plant promoter” is a promoter capable of initiating transcription in plant cells.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” means conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence. The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

“Silencing,” as used herein with respect to a target gene, refers generally to the suppression of levels of mRNA or protein/enzyme expressed by the target gene, and/or the level of the enzyme activity or protein functionality.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

“Suppression DNA construct” is a recombinant DNA construct which when transformed or stably integrated into the genome of the plant, results in “silencing” of a target gene in the plant. The target gene may be endogenous or transgenic to the plant.

The terms “suppress”, “suppressed”, “suppression”, “suppressing” and “silencing”, are used interchangeably herein and include lowering, reducing, declining, decreasing, inhibiting, eliminating or preventing.

“Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches and the like.

The terms “target site”, “target sequence”, “target DNA”, “target locus”, “genomic target site”, “genomic target sequence”, and “genomic target locus” are used interchangeably herein and refer to a polynucleotide sequence in the genome (including chloroplast and mitochondrial DNA) of a cell at which a double-strand break is induced in the cell genome. The target site can be an endogenous site in the genome of a cell or organism, or alternatively, the target site can be heterologous to the cell or organism and thereby not be naturally occurring in the genome, or the target site can be found in a heterologous genomic location compared to where it occurs in nature. As used herein, terms “endogenous target sequence” and “native target sequence” are used interchangeably herein to refer to a target sequence that is endogenous or native to the genome of a cell or organism and is at the endogenous or native position of that target sequence in the genome of a cell or organism. Cells include plant cells as well as plants and seeds produced by the methods described herein.

“Tissue-specific promoter” and “tissue-preferred promoter” are used interchangeably, and refer to a promoter that is expressed predominantly but not necessarily exclusively in one tissue or organ, but that may also be expressed in one specific cell.

“Transgenic” generally refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Transgenic plant” includes reference to a plant which comprises within its genome a heterologous polynucleotide.

“Transgenic plant” also includes reference to plants which comprise more than one heterologous polynucleotide within their genome. Each heterologous polynucleotide may confer a different trait to the transgenic plant.

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

The term “under stringent conditions” means that two sequences hybridize under moderately or highly stringent conditions.

The term “univalent” means that a chromosome that is not paired with its homologous chromosome during synapsis.

The term “bivalents” means that two homologous chromosomes paired at meiosis I.

### Introduction

Described herein are maize Spo11, Rec8, OSD1-1 and OSD1-3 polynucleotides and polypeptides. The term “Spo11 polypeptide” refers to one or more Spo11 amino acid sequences. The term is also inclusive of fragments, variants, homologs, alleles or precursors (e.g., preproproteins or proproteins) thereof. A “Spo11 protein” comprises a Spo11 polypeptide. Unless otherwise stated, the term “Spo11 nucleic acid” means a nucleic acid comprising a polynucleotide (“Spo11 polynucleotide”) encoding a Spo11 polypeptide. As used herein, Spo11 activity refers to the Spo11 protein's ability to promote homologous pairing and double strand breaks (DSBs) that result in recombination between homologous chromosomes during meiosis in plants. As shown in Example 1, a maize spo11 mutant resulted in the formation of univalent rather than bivalents at meiosis I, which indicates that Spo11 is involved in homologous pairing of chromosomes during meiosis.

The term “Rec8 polypeptide” refers to one or more Rec8 amino acid sequences. The term is also inclusive of fragments, variants, homologs, alleles or precursors (e.g., preproproteins or proproteins) thereof. A “Rec8 protein” comprises a Rec8 polypeptide. Unless otherwise stated, the term “Rec8 nucleic acid” means a nucleic acid comprising a polynucleotide (“Rec8 polynucleotide”) encoding a Rec8 polypeptide. The terms Rec8 and Afd1 used herein interchangeably. As described in Example 1, a maize Rec8 mutant showed chromosome disorientation at meiosis I, resulting in unbalanced meiotic products at meiosis II. As used herein, Rec8 activity refers to the Rec8 protein's ability to bind sister chromatids of homologous chromosomes for proper separation during meiosis 1 and to orient kinetochores during meiosis in plants.

When maize was disrupted for both spo11/rec8 the double mutant had a mitotic like first division, rather than a meiosis division. See, for example, Example 1.

The term “OSD1-1A polypeptide” refers to one or more OSD1-1A amino acid sequences. The term is also inclusive of fragments, variants, homologs, alleles or precursors (e.g., preproproteins or proproteins) thereof. A “OSD1-1A protein” comprises a OSD1-1A polypeptide. Unless otherwise stated, the term “OSD1-1A nucleic acid” means a nucleic acid comprising a polynucleotide (“OSD1-1A polynucleotide”) encoding a OSD1-1A polypeptide. As used herein, OSD1-1A activity refers to the OSD1-1A protein's ability to promote the progression of meiosis II division during meiosis in plants and produce reduced gametes.

The term “OSD1-3A polypeptide” refers to one or more OSD1-3A amino acid sequences. The term is also inclusive of fragments, variants, homologs, alleles or precursors (e.g., preproproteins or proproteins) thereof. A “OSD1-3A protein” comprises a OSD1-3A polypeptide. Unless otherwise stated, the term “OSD1-3A nucleic acid” means a nucleic acid comprising a polynucleotide (“OSD1-3 polynucleotide”) encoding a OSD1-3A polypeptide. As used herein, OSD1-3A activity refers to the OSD1-3A protein's ability to promote the progression of meiosis II division during meiosis in plants and produce reduced gametes. Suppression of both OSD1-1A and OSD1-3A together in maize may result in the early exit of meiosis and non-reduced gametes. In species other than maize, suppression of one OSD1 gene may be sufficient to disrupt meiosis and reduction of gametes, for example, in rice. Use of amiRNA for both maize OSD1-1A and OSD1-3A in the maize rec8/spo11 mutant described in Example 1 resulted in the production of both male and female reduced and non-reduced gametes, indicating a role for maize OSD1-1A and OSD1-3A together in meiosis II division.

The suppression of Spo11, Rec8, OSD1-1A, and OSD1-3A polynucleotides and polypeptides or combinations thereof thus provide an opportunity to manipulate sexual reproduction and/or alter gametophyte and seed development. Suppression of Spo11, Rec8 and OSD1-1A, and OSD1-3A polynucleotides and polypeptides or combinations thereof may be used to produce clonal, non-reduced and non-recombined male and/or female gametes. In certain embodiments, the methods include suppressing an endogenous Spo11, Rec8 and OSD1-1A, and OSD1-3A polynucleotides and polypeptides or combinations thereof in a plant or plant cell. In certain embodiments, the plant cell is a microspore mother cell (which gives rise to haploid microspores), megaspore mother cell (which gives rise to a haploid megaspore) or any other plant cell where the gene, e.g. Spo11, Rec8 and OSD1-1A, and OSD1-3A, is expressed. Embodiments include isolated polynucleotides and polypeptides, recombinant DNA constructs useful for modifying meiosis in plant cells, asexual reproductive development and/or producing clonal progeny, including seeds and plants, that are non-reduced and non-recombined with respect to gametes.

The present disclosure includes the following isolated polynucleotides and polypeptides:

An isolated Spo11 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:16; or (ii) a full complement of the nucleic acid sequence of (i), wherein the full complement and the nucleic acid sequence of (i) consist of the same number of nucleotides and are 100% complementary.

An isolated Spo11 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:16.

An isolated Spo11 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO:13, 14, 15, or 19 and combinations thereof; or (ii) a full complement of the nucleic acid sequence of (i).

An isolated Spo11 polynucleotide comprising a nucleotide sequence, wherein the nucleotide sequence is hybridizable under stringent conditions with a DNA molecule comprising the full complement of SEQ ID NO:13, 14, 15, or 19. The isolated Spo11 protein of the present disclosure may also be a protein which is encoded by a nucleic acid comprising a nucleotide sequence hybridizable under stringent conditions with the complementary strand of the nucleotide sequence of SEQ ID NO:13, 14, or 15.

An isolated Spo11 polynucleotide comprising a nucleotide sequence, wherein the nucleotide sequence is derived from SEQ ID NO:13, 14, 15, or 19 by alteration of one or more nucleotides by at least one method selected from the group consisting of: deletion, substitution, addition and insertion.

An isolated Spo11 polynucleotide comprising a nucleotide sequence, wherein the nucleotide sequence corresponds to an allele of SEQ ID NO:13, 14, 15, or 19.

Any of the foregoing isolated polynucleotides may be utilized in any recombinant DNA constructs (including suppression DNA constructs) of the present disclosure.

An isolated Rec8 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:12; or (ii) a full complement of the nucleic acid sequence of (i), wherein the full complement and the nucleic acid sequence of (i) consist of the same number of nucleotides and are 100% complementary.

An isolated Rec8 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:12.

An isolated Rec8 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO:9, 10, 11 or 20 and combinations thereof; or (ii) a full complement of the nucleic acid sequence of (i).

An isolated Rec8 polynucleotide comprising a nucleotide sequence, wherein the nucleotide sequence is hybridizable under stringent conditions with a DNA molecule comprising the full complement of SEQ ID NO:9, 10, 11 or 20. The isolated Rec8 protein of the present disclosure may also be a protein which is encoded by a nucleic acid comprising a nucleotide sequence hybridizable under stringent conditions with the complementary strand of the nucleotide sequence of SEQ ID NO: 9, 10, or 11.

An isolated Rec8 polynucleotide comprising a nucleotide sequence, wherein the nucleotide sequence is derived from SEQ ID NO: 9, 10, 11 or 20 by alteration of one or more nucleotides by at least one method selected from the group consisting of: deletion, substitution, addition and insertion.

An isolated Rec8 polynucleotide comprising a nucleotide sequence, wherein the nucleotide sequence corresponds to an allele of SEQ ID NO:9, 10, 11 or 20.

Any of the foregoing isolated polynucleotides may be utilized in any recombinant DNA constructs (including suppression DNA constructs) of the present disclosure.

An isolated OSD1-1 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:4; or (ii) a full complement of the nucleic acid sequence of (i), wherein the full complement and the nucleic acid sequence of (i) consist of the same number of nucleotides and are 100% complementary.

An isolated OSD1-1A polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:4.

An isolated OSD1-1A polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO:1, 2, 3 or 21 and combinations thereof; or (ii) a full complement of the nucleic acid sequence of (i). Any of the foregoing isolated polynucleotides may be utilized in any recombinant DNA constructs (including suppression DNA constructs) of the present disclosure.

An isolated OSD1-1A polynucleotide comprising a nucleotide sequence, wherein the nucleotide sequence is hybridizable under stringent conditions with a DNA molecule comprising the full complement of SEQ ID NO:1, 2, 3 or 21. The isolated OSD1-1A protein of the present disclosure may also be a protein which is encoded by a nucleic acid comprising a nucleotide sequence hybridizable under stringent conditions with the complementary strand of the nucleotide sequence of SEQ ID NO:1, 2, or 3.

An isolated OSD1-1A polynucleotide comprising a nucleotide sequence, wherein the nucleotide sequence is derived from SEQ ID NO:1, 2, 3 or 21 by alteration of one or more nucleotides by at least one method selected from the group consisting of: deletion, substitution, addition and insertion.

An isolated OSD1-1A polynucleotide comprising a nucleotide sequence, wherein the nucleotide sequence corresponds to an allele of SEQ ID NO:1, 2, 3 or 21.

Any of the foregoing isolated polynucleotides may be utilized in any recombinant DNA constructs (including suppression DNA constructs) of the present disclosure.

An isolated OSD1-3A polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:8; or (ii) a full complement of the nucleic acid sequence of (i), wherein the full complement and the nucleic acid sequence of (i) consist of the same number of nucleotides and are 100% complementary.

An isolated OSD1-3A polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:8.

An isolated OSD1-3A polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 5, 6, 7, or 22 and combinations thereof; or (ii) a full complement of the nucleic acid sequence of (i). Any of the foregoing isolated polynucleotides may be utilized in any recombinant DNA constructs (including suppression DNA constructs) of the present disclosure.

An isolated OSD1-3A polynucleotide comprising a nucleotide sequence, wherein the nucleotide sequence is hybridizable under stringent conditions with a DNA molecule comprising the full complement of SEQ ID NO:5, 6, 7, or 22. The isolated OSD1-3A protein of the present disclosure may also be a protein which is encoded by a nucleic acid comprising a nucleotide sequence hybridizable under stringent conditions with the complementary strand of the nucleotide sequence of SEQ ID NO:5, 6, or 7.

An isolated OSD1-3A polynucleotide comprising a nucleotide sequence, wherein the nucleotide sequence is derived from SEQ ID NO:5, 6, 7, or 22 by alteration of one or more nucleotides by at least one method selected from the group consisting of: deletion, substitution, addition and insertion.

An isolated OSD1-3A polynucleotide comprising a nucleotide sequence, wherein the nucleotide sequence corresponds to an allele of SEQ ID NO:5, 6, 7, or 22.

Nucleic acid molecules that are fragments of these nucleic acid sequences encoding Spo11, Rec8, OSD1-1A or OSD1-3A polypeptides are also encompassed by the embodiments. “Fragment” as used herein refers to a portion of the nucleic acid sequence encoding a Spo11, Rec8, OSD1-1A or OSD1-3A polypeptide. A fragment of a nucleic acid sequence may encode a biologically active portion of Spo11, Rec8, OSD1-1A or OSD1-3A polypeptide or it may be a fragment that can be used as a hybridization probe or PCR primer using methods disclosed below. Nucleic acid molecules that are fragments of a nucleic acid sequence encoding a Spo11, Rec8, OSD1-1A or OSD1-3A polypeptide comprise at least about 150, 180, 210, 240, 270, 300, 330, 360, 400, 450, 500, 550, 600, 650, 700, 750, 800, 850, 900, 950, 1000, 1050, 1100, 1150, 1200, 1250, 1300, 1350 or contiguous nucleotides or up to the number of nucleotides present in a full-length nucleic acid sequence encoding a Spo11, Rec8, OSD1-1A or OSD1-3A polypeptide disclosed herein, depending upon the intended use. “Contiguous nucleotides” is used herein to refer to nucleotide residues that are immediately adjacent to one another. Fragments of the nucleic acid sequences of the embodiments will encode protein fragments that retain the biological activity of the Spo11, Rec8, OSD1-1A or OSD1-3A polypeptide and, hence, retain its respective Spo11, Rec8, OSD1-1A or OSD1-3A activity.

Any of the foregoing isolated polynucleotides may be utilized in any recombinant DNA constructs (including suppression DNA constructs) of the present disclosure. Accordingly, in certain embodiments, compositions (such as plants or seeds) comprising these isolated Spo11, Rec8, OSD1-1A, and/or OSD1-3A polynucleotides and polypeptides or combinations thereof are provided. Recombinant DNA constructs expressing the isolated Spo11, Rec8, OSD1-1A, and/or OSD1-3A polynucleotides or combinations thereof are also provided.

In certain embodiments, suppression Spo11, Rec8, OSD1-1A, and/or OSD1-3A polynucleotides or combinations thereof are disclosed. Also included herein are recombinant DNA constructs expressing the suppression Spo11, Rec8, OSD1-1A, and/or OSD1-3A polynucleotides or combinations thereof. Methods may include the use of any of these isolated or suppression Spo11, Rec8, OSD1-1A, and OSD1-3A polynucleotides, and the recombinant DNA constructs comprising them. Additionally, the present disclosure relates to a vector containing the recombinant DNA construct. Further, the vector containing the recombinant expression cassette or construct can facilitate the transcription and translation of the nucleic acid in a host cell. The present disclosure also relates to the host cells able to express the polynucleotides disclosed herein. A number of host cells could be used, such as but not limited to, microbial, mammalian, plant or insect.

Methods are provided to suppress the activity of an endogenous Spo11, Rec8, OSD1-1A, and/or OSD1-3A polynucleotide or polypeptide, or combinations thereof in a plant cell. In some aspects, suppressing activity also includes suppressing the expression level of the Spo11, Rec8, OSD1-1A, and OSD1-3A polynucleotide or polypeptide. Any suitable method or technique may be used. One of ordinary skill in the art would readily recognize a suitable control or reference to be utilized when assessing or measuring expression level or activity of Spo11, Rec8, OSD1-1A, and/or OSD1-3A polynucleotide or polypeptide in any embodiment of the present disclosure in which a control or reference is utilized (e.g., compositions or methods as described herein). For example, by way of non-limiting illustrations, a plant or plant cell comprising a modified Spo11, Rec8, OSD1-1A, and/or OSD1-3A polynucleotide or polypeptide; or Spo11, Rec8, OSD1-1A, and/or OSD1-3A suppression polynucleotide would be typically measured relative to a plant or plant cell not comprising the modified Spo11, Rec8, OSD1-1A, and/or OSD1-3A polynucleotide or polypeptide, or the Spo11, Rec8, OSD1-1A, and/or OSD1-3A suppression polynucleotide as the control or reference plant or plant cell. In some examples, the control is a wild type plant or cell. One skilled in the art will be able to determine Spo11, Rec8, OSD1-1A, and/or OSD1-3A expression level or activity using assays, such as PCR, Northern, and Western blot assays.

In certain embodiments, an endogenous gene of Spo11, Rec8, OSD1-1A, and/or OSD1-3A, or combinations thereof may be modified in a plant cell. Accordingly, the present disclosure is directed to a plant or plant cells comprising the modified Spo11, Rec8, OSD1-1A, and/or OSD1-3A. Another embodiment is the seed from the modified plant. In some embodiments, the modification gene is different than the gene in its native form in composition and/or genomic locus through deliberate human intervention. Methods and techniques to modify or alter Spo11, Rec8, OSD1-1A, and/or OSD1-3A genes, or combinations thereof are provided herein. In some examples, this includes altering the host plant native DNA sequence or a pre-existing recombinant sequence including regulatory elements, coding and/or non-coding sequences. In certain examples of genome editing approaches, endonucleases including but not limited to Cas endonuclease and guide RNA, are employed to delete, edit, insert or substitute all or part of a plant cell's endogenous Spo11, Rec8, OSD1-1A, and/or OSD1-3A genes or combinations thereof to suppress the respective endogenous Spo11, Rec8, OSD1-1A, and OSD1-3A activity. In some aspects, suppressing activity also includes suppressing the expression level of the Spo11, Rec8, OSD1-1A, and/or OSD1-3A polynucleotide or polypeptide.

In certain embodiments, the plant cell's endogenous Spo11, Rec8, or OSD1-1A, and/or OSD1-3A genes may be modified using genome editing technologies including but not limited to meganucleases, zinc finger nucleases, transcription activator-like effector nucleases (TALENS), CRISPR-Cas9 and RNA-guided endonucleases. Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex.

Endonucleases also include meganucleases, also known as homing endonucleases (HEases). Like restriction endonucleases, HEases bind and cut at a specific recognition site. However, the recognition sites for meganucleases are typically longer, about 18 bp or more. (See patent publication WO2012/129373 filed on Mar. 22, 2012). One step in the recombination process involves polynucleotide cleavage at or near the meganuclease recognition site. This cleaving activity can be used to produce a double-strand break. For reviews of site-specific recombinases and their recognition sites, see, Sauer (1994) Curr. Op. Biotechnol. 5:521-7; and Sadowski (1993) FASEB 7:760-7. In some examples the recombinase is from the Integrase or Resolvase families.

TAL effector nucleases are a class of sequence-specific nucleases that can be used to make double-strand breaks at specific target sequences in the genome of a plant or other organism. (Miller et al. (2011) Nature Biotechnology 29:143-148). Zinc finger nucleases (ZFNs) are engineered double-strand-break-inducing agents comprised of a zinc finger DNA binding domain and a double-strand-break-inducing agent domain. Recognition site specificity is conferred by the zinc finger domain, which typically comprises two, three, or four zinc fingers, for example having a C2H2 structure; however other zinc finger structures are known and have been engineered. Zinc finger domains are amenable for designing polypeptides which specifically bind a selected polynucleotide recognition sequence. Each zinc finger recognizes three consecutive base pairs in the target DNA. For example, a 3-finger domain recognizes a sequence of 9 contiguous nucleotides; with a dimerization requirement of the nuclease, two sets of zinc finger triplets are used to bind an 18-nucleotide recognition sequence.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times-also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

Cas gene relates to a gene that is generally coupled, associated or close to or in the vicinity of flanking CRISPR loci. The terms “Cas gene”, “CRISPR-associated (Cas) gene” are used interchangeably herein. A comprehensive review of the Cas protein family is presented in Haft et al. (2005) Computational Biology, PLOS Comput Biol 1(6): e60. doi:10.1371/journal.pcbi.0010060. As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

Cas endonuclease relates to a Cas protein encoded by a Cas gene, wherein said Cas protein is capable of introducing a double strand break into a DNA target sequence. The Cas endonuclease is guided by a guide polynucleotide to recognize and optionally introduce a double strand break at a specific target site into the genome of a cell (U.S. Provisional Application No. 62/023,239, filed Jul. 11, 2014). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

The Cas endonuclease gene can be Cas9 endonuclease, or a functional fragment thereof, such as but not limited to, Cas9 genes listed in SEQ ID NOs: 462, 474, 489, 494, 499, 505, and 518 of WO2007/025097 published Mar. 1, 2007. The Cas endonuclease gene can be a plant, maize or soybean optimized Cas9 endonuclease, such as but not limited to a plant codon optimized Streptococcus pyogenes Cas9 gene that can recognize any genomic sequence of the form N(12-30)NGG. The Cas endonuclease can be introduced directly into a cell by any method known in the art, for example, but not limited to transient introduction methods, transfection and/or topical application.

As used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

As used herein, the term “guide polynucleotide”, relates to a polynucleotide sequence that can form a complex with a Cas endonuclease and enables the Cas endonuclease to recognize and optionally cleave a DNA target site (U.S. Provisional Application No. 62/023,239, filed Jul. 11, 2014). The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotride that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In one embodiment, the size of the fragment of the cRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides In one embodiment, the RNA that guides the RNA/Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). In one embodiment of the disclosure, the single guide RNA comprises a cRNA or cRNA fragment and a tracrRNA or tracrRNA fragment of the type II/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In certain embodiments the nucleotide sequence to be modified can be a regulatory sequence such as a promoter, wherein the editing of the promoter comprises replacing the promoter (also referred to as a “promoter swap” or “promoter replacement”) or promoter fragment with a different promoter (also referred to as replacement promoter) or promoter fragment (also referred to as replacement promoter fragment), wherein the promoter replacement results in any one of the following or any combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression in the same cell layer or other cell layer (such as but not limiting to extending the timing of gene expression in the tapetum of maize anthers; see e.g. U.S. Pat. No. 5,837,850 issued Nov. 17, 1998), a mutation of DNA binding elements and/or deletion or addition of DNA binding elements. The promoter (or promoter fragment) to be modified can be a promoter (or promoter fragment) that is endogenous, artificial, pre-existing, or transgenic to the cell that is being edited. The replacement promoter (or replacement promoter fragment) can be a promoter (or promoter fragment) that is endogenous, artificial, pre-existing, or transgenic to the cell that is being edited.

Promoter elements to be inserted can be, but are not limited to, promoter core elements (such as, but not limited to, a CAAT box, a CCAAT box, a Pribnow box, a and/or TATA box, translational regulation sequences and/or a repressor system for inducible expression (such as TET operator repressor/operator/inducer elements, or SulphonylUrea (Su) repressor/operator/inducer elements. The dehydration-responsive element (DRE) was first identified as a cis-acting promoter element in the promoter of the drought-responsive gene rd29A, which contains a 9 bp conserved core sequence, TACCGACAT (Yamaguchi-Shinozaki, K., and Shinozaki, K. (1994) Plant Cell 6, 251-264). Insertion of DRE into an endogenous promoter may confer a drought inducible expression of the downstream gene. Another example is ABA-responsive elements (ABREs) which contain a (C/T)ACGTGGC consensus sequence found to be present in numerous ABA and/or stress-regulated genes (Busk P. K., Pages M.(1998) Plant Mol. Biol. 37:425-435). Insertion of 35S enhancer or MMV enhancer into an endogenous promoter region will increase gene expression (U.S. Pat. No. 5,196,525). The promoter (or promoter element) to be inserted can be a promoter (or promoter element) that is endogenous, artificial, pre-existing, or transgenic to the cell that is being edited.

In certain embodiments, a suppression polynucleotide sequence is capable of suppressing the activity of the endogenous Spo11, Rec8, OSD1-1A, and/or OSD1-3A polypeptide(s) or combinations thereof disclosed elsewhere herein. In some aspects, suppressing activity includes suppressing the expression level of the Spo11, Rec8, OSD1-1A, and/or OSD1-3A polynucleotide or polypeptide. Suppression polynucleotides may be introduced into a plant cell to suppress endogenous gene(s) of Spo11, Rec8, OSD1-1A, and/or OSD1-3A, or combinations thereof using any suitable technique or method or mechanism that results in the suppression of Spo11, Rec8, OSD1-1A, and/or OSD1-3A activity. In certain embodiments, Spo11, Rec8, OSD1-1A, and/or OSD1-3A or combinations thereof are suppressed using anti-sense, cosuppression, viral-suppression, hairpin suppression, stem-loop suppression, RNAi-based approaches, and small RNA-based approaches. In certain examples, genome editing approaches, including but not limited to, Cas endonuclease and guide RNA, are employed to introduce into a plant cell's genome polynucleotides that suppress Spo11, Rec8, OSD1-1A, and/or OSD1-3A expression and/or activity. Examples of such Spo11, Rec8, OSD1-1A, and/or OSD1-3A suppression polynucleotides that may be used to suppress the expression of Spo11, Rec8, OSD1-1A, and/or OSD1-3A are provided herein elsewhere.

In some embodiments, Spo11, Rec8, OSD1-1A, and/or OSD1-3A suppression polynucleotides, or combinations thereof comprise polynucleotides for cosuppression. In certain embodiments for cosuppression, the polynucleotide is designed to express an RNA molecule corresponding to all or part of a messenger RNA encoding a Spo11, Rec8, OSD1-1A, and/or OSD1-3A polypeptide in the “sense” orientation.

Over expression of the RNA molecule can result in reduced expression of the native gene. In certain embodiments for cosuppression, the cosuppression polynucleotide linked to a heterologous promoter and expressed from a cosuppression expression cassette. Plant cells may be transformed with the cosuppression polynucleotide or cosuppression expression cassette and screened to identify those that show the greatest suppression of Spo11, Rec8, OSD1-1A, and/or OSD1-3A polypeptide expression.

The polynucleotide used for cosuppression may correspond to all or part of the sequence encoding the Spo11, Rec8, OSD1-1A, and/or OSD1-3A polypeptide, all or part of the 5′ and/or 3′ untranslated region of an Spo11, Rec8, OSD1-1A, and/or OSD1-3A polypeptide transcript or all or part of both the coding sequence and the untranslated regions of a transcript encoding an Spo11, Rec8, OSD1-1A, and/or OSD1-3A polypeptide. In some embodiments where the polynucleotide comprises all or part of the coding region for the Spo11, Rec8, OSD1-1A, and/or OSD1-3A polypeptide, the expression cassette is designed to eliminate the start codon of the polynucleotide so that no protein product will be translated.

Cosuppression may be used to suppress the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

In some embodiments, suppression of the expression of the Spo11, Rec8, OSD1-1A, and/or OSD1-3A polypeptide may be obtained by antisense suppression. For antisense suppression, the expression cassette is designed to express an RNA molecule complementary to all or part of a messenger RNA encoding the Spo11, Rec8, OSD1-1A, and/or OSD1-3A polypeptide. Over expression of the antisense RNA molecule can result in reduced expression of the native gene. Accordingly, multiple plant lines transformed with the antisense suppression expression cassette are screened to identify those that show the greatest inhibition of a Spo11, Rec8, OSD1-1A, and/or OSD1-3A polypeptide expression.

The polynucleotide for use in antisense suppression may correspond to all or part of the complement of the sequence encoding the Spo11, Rec8, OSD1-1A, and/or OSD1-3A polypeptide, all or part of the complement of the 5′ and/or 3′ untranslated region of the Spo11, Rec8, OSD1-1A, and/or OSD1-3A transcript or all or part of the complement of both the coding sequence and the untranslated regions of a transcript encoding the Spo11, Rec8, OSD1-1A, and/or OSD1-3A polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene, for example, Spo11, Rec8, OSD1-1A, and/or OSD1-3A. Generally, sequences of at least 50 nucleotides, 100 nucleotides, 200 nucleotides, 300, 400, 450, 500, 550, 600 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

In certain embodiments, the present disclosure includes suppression Spo11, Rec8, OSD1-1A and OSD1-3A polynucleotides for use in the methods described herein.

A Spo11 suppression polynucleotide may comprise all or part of: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:16, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with said all or part of a sense strand or antisense strand from which said region is derived, and wherein said target gene of interest encodes a Spo11 polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 13, 14, 15, or 19, and combinations thereof, or (ii) a full complement of the nucleic acid sequence of (c)(i). In certain embodiments the Spo11 suppression polynucleotide is operably liked to at least one heterologous regulatory sequence (e.g., a promoter functional in a plant). For example, the Spo11 suppression polynucleotide may be inserted via gene editing or other approach into a plant chromosome and expressed so that it suppresses endogenous Spo11 expression or activity. The Spo11 suppression polynucleotide may be operably liked to at least one heterologous regulatory sequence (e.g., a promoter functional in a plant). In some instances, the Spo11 suppression polynucleotide may be operably liked to at least one heterologous regulatory sequence and expressed from a suppression DNA construct.

A Rec8 suppression polynucleotide may comprise all or part of: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:12, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with said all or part of a sense strand or antisense strand from which said region is derived, and wherein said target gene of interest encodes a Rec8 polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 9, 10, 11 or 20, and combinations thereof, or (ii) a full complement of the nucleic acid sequence of (c) (i). In certain embodiments the Rec8 suppression polynucleotide is operably liked to at least one heterologous regulatory sequence (e.g., a promoter functional in a plant). For example, the Rec8 suppression polynucleotide may be inserted via gene editing or other approach into a plant chromosome and expressed so that it suppresses endogenous Rec8 expression or activity. The Rec8 suppression polynucleotide may be operably liked to at least one heterologous regulatory sequence (e.g., a promoter functional in a plant). In some instances, the Rec8 suppression polynucleotide may be operably liked to at least one heterologous regulatory sequence and expressed from a suppression DNA construct.

A OSD1-1A suppression polynucleotide may comprise all or part of: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:4, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with said all or part of a sense strand or antisense strand from which said region is derived, and wherein said target gene of interest encodes a OSD1-1A polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 1, 2, 3 or 21, and combinations thereof, or (ii) a full complement of the nucleic acid sequence of (c)(i). In certain embodiments the OSD1-1A suppression polynucleotide is operably liked to at least one heterologous regulatory sequence (e.g., a promoter functional in a plant). For example, the OSD1-1A suppression polynucleotide may be inserted via gene editing or other approach into a plant chromosome and expressed so that it suppresses endogenous OSD1-1A expression or activity. The OSD1-1A suppression polynucleotide may be operably liked to at least one heterologous regulatory sequence (e.g., a promoter functional in a plant). In some instances, the OSD1-1A suppression polynucleotide may be operably liked to at least one heterologous regulatory sequence and expressed from a suppression DNA construct.

A OSD1-3A suppression polynucleotide may comprise all or part of: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO:8, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with said all or part of a sense strand or antisense strand from which said region is derived, and wherein said target gene of interest encodes a OSD1-3A polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO:5, 6, 7 or 22, and combinations thereof, or (ii) a full complement of the nucleic acid sequence of (c)(i). In certain embodiments the OSD1-3A suppression polynucleotide is operably liked to at least one heterologous regulatory sequence (e.g., a promoter functional in a plant). For example, the OSD1-3A suppression polynucleotide may be inserted via gene editing or other approach into a plant chromosome and expressed so that it suppresses endogenous OSD1-3A expression or activity. The OSD1-3A suppression polynucleotide may be operably liked to at least one heterologous regulatory sequence (e.g., a promoter functional in a plant). In some instances, the OSD1-3A suppression polynucleotide may be operably liked to at least one heterologous regulatory sequence and expressed from a suppression DNA construct.

In some embodiments, suppression of the expression of a Spo11, Rec8, OSD1-1A, and/or OSD1-3A polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of a Spo11, Rec8, OSD1-1A, and/or OSD1-3A polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

In some embodiments, inhibition of the expression of a Spo11, Rec8, OSD1-1A, and/or OSD1-3A polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295, and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene, for example, Spo11, Rec8, OSD1-1A, and/or OSD1-3A. Thus, it is the loop region that determines the specificity of the RNA interference. See, for example, WO 2002/00904, Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the Spo11, Rec8, OSD1-1A, and/or OSD1-3A polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

In some embodiments, the polynucleotide expressed by the expression cassette of the disclosure is catalytic RNA or has ribozyme activity specific for the messenger RNA of the Spo11, Rec8, OSD1-1A, and/or OSD1-3A polypeptide. Thus, the polynucleotide causes the degradation of the endogenous messenger RNA, resulting in reduced expression of the Spo11, Rec8, OSD1-1A, and/or OSD1-3A polypeptide. This method is described, for example, in U.S. Pat. No. 4,987,071, herein incorporated by reference.

RNA interference generally refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

In some embodiments, suppression of the expression of a Spo11, Rec8, OSD1-1A, and/or OSD1-3A polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference.

In one embodiment, there is provided a method for the suppression of a target sequence comprising introducing into a cell a nucleic acid construct encoding a miRNA substantially complementary to the target. In certain embodiments, the target is the Spo11, Rec8, OSD1-1A, and/or OSD1-3A polynucleotides disclosed herein. In certain embodiments, maize OSD1-1A and OSD1-3A artificial microRNA (amiRNA) target sequences are those found in SEQ ID NO:17 and SEQ ID NO:18 respectively. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence. In certain embodiments, the target is the Spo11, Rec8, OSD1-1A, and/or OSD1-3A polynucleotides disclosed herein.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

In certain embodiments, for miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of Spo11, Rec8, OSD1-1A, and/or OSD1-3A expression, the 22-nucleotide sequence is selected from a Spo11, Rec8, OSD1-1A, and/or OSD1-3A transcript sequence and contains 22 nucleotides of said Spo11, Rec8, OSD1-1A, and/or OSD1-3A sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

In one embodiment, the polynucleotide encodes a zinc finger protein that binds to a gene encoding Spo11, Rec8, OSD1-1A, and/or OSD1-3A polypeptide, resulting in reduced expression of the cognate Spo11, Rec8, OSD1-1A, OSD1-3A gene. In particular embodiments, the zinc finger protein binds to a regulatory region of the Spo11, Rec8, OSD1-1A, and/or OSD1-3A gene. In other embodiments, the zinc finger protein binds to a messenger RNA encoding a Spo11, Rec8, OSD1-1A, and/or OSD1-3A polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355, each of which is herein incorporated by reference.

In some embodiments, the polynucleotide encodes an antibody that binds to at least one Spo11, Rec8, OSD1-1A, and/or OSD1-3A polypeptide, or combinations thereof and suppresses the Spo11, Rec8, OSD1-1A, OSD1-3A activity of the respective Spo11, Rec8, OSD1-1A, OSD1-3A polypeptide. In another embodiment, the binding of the antibody results in increased turnover of the antibody-SPO11, REC8, OSD1-1A, and/or OSD1-3A complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

In some embodiments, the activity of a Spo11, Rec8, OSD1-1A, and/or OSD1-3A polypeptide is suppressed by disrupting the gene encoding the Spo11, Rec8, OSD1-1A, and/or OSD1-3A polypeptide, for example, using any method known in the art, including but not limited to genome editing approaches. In certain embodiments, the Spo11, Rec8, OSD1-1A, and/or OSD1-3A gene is disrupted by transposon tagging. In another embodiment, the Spo11, Rec8, OSD1-1A, and/or OSD1-3A gene is disrupted by mutagenizing plants using random or targeted mutagenesis, such as or TUSC mutations, and selecting for plants that have reduced Spo11, Rec8, OSD1-1A, and/or OSD1-3A activity, for example, expression level, or combinations thereof. Additional methods for suppressing the expression of an endogenous Spo11, Rec8, OSD1-1A, and/or OSD1-3A polypeptide in plants include ethyl methanesulfonate-induced mutagenesis and deletion mutagenesis. In addition, a fast and automatable method for screening for chemically induced mutations, TILLING (Targeting Induced Local Lesions In Genomes), using denaturing HPLC or selective endonuclease digestion of selected PCR products is also applicable may also be used. See, McCallum, et al., (2000) Nat. Biotechnol. 18:455-457, herein incorporated by reference.

As described elsewhere herein, the suppression polynucleotides can be introduced in a variety of ways. As discussed herein, one of skill will recognize the appropriate promoter to use to for use in driving expression of Spo11, Rec8, OSD1-1A, and/or OSD1-3A polynucleotides or suppression polynucleotides in a plant or plant cell. The suppression polynucleotides can be expressed in a specific manner, for example, using constitutive, inducible or tissue-preferred or developmentally regulated promoters that are discussed elsewhere herein. In one embodiment, the suppression polynucleotides is operably linked to a Spo11, Rec8, OSD1-1A or OSD1-3A promoter, variant or fragment thereof. The promoter may be the native promoter for the endogenous Spo11, Rec8, OSD1-1A or OSD1-3A gene, or heterologous to the endogenous Spo11, Rec8, OSD1-1A or OSD1-3A gene, for example, from another species or crop. In certain embodiments, the suppression polynucleotides are expressed in a plant cell undergoing meiosis.

In certain embodiments, the disclosure includes a method for obtaining a maize plant with a modified endogenous Spo11 polynucleotide sequence. In certain aspects, the modified endogenous Spo11 disrupts the homologous pairing and subsequent recombination of chromosomes during meiosis in a plant cell. In certain aspects, the activity of the endogenous Spo11 polynucleotide or polypeptide is suppressed using genome editing approaches, for example, by genetically modifying the endogenous Spo11. In some approaches, the suppression results from a nucleotide modification of the endogenous maize Spo11 polynucleotide sequences. In some embodiments, the nucleotide modification is a deletion, addition, or substitution of one or more nucleotides. In certain embodiments, the suppression results from an amino modification of the endogenous Spo11 polypeptide sequence. In some embodiments, the amino acid modification is a deletion, addition, or substitution of one or more amino acids. In certain aspects, the activity of the endogenous Spo11 polynucleotide or polypeptide is suppressed using RNA-based silencing approaches, for example, antisense, microRNA, RNAi, or hairpin molecule, described elsewhere herein. In certain embodiments, the suppression of Spo11 is a knock-out of the gene.

In certain embodiments, the disclosure includes a method for obtaining a maize plant with a modified endogenous Rec8 polynucleotide sequence. In certain aspects, the modified endogenous Rec8 disrupts the orientation of kinetochores and subsequent random distribution of chromatids during meiosis II in a plant cell. In certain aspects, the activity of the endogenous Rec8 polynucleotide or polypeptide is suppressed using genome editing approaches, for example, by genetically modifying the endogenous Rec8. In some approaches, the suppression results from a nucleotide modification of the endogenous maize Rec8 polynucleotide sequences. In some embodiments, the nucleotide modification is a deletion, addition, or substitution of one or more nucleotides. In certain embodiments, the suppression results from an amino of modification of the endogenous Rec8 polypeptide sequence. In some embodiments, the amino acid modification is a deletion, addition, or substitution of one or more amino acids. In certain aspects, the activity of the endogenous Rec8 polynucleotide or polypeptide is suppressed using RNA-based silencing approaches, for example, antisense, microRNA, RNAi, or hairpin molecule, described elsewhere herein. In certain embodiments, the suppression of Rec8 is a knock-out of the gene.

In certain embodiments, the disclosure includes a method for obtaining a maize plant with a modified endogenous OSD1-1A polynucleotide sequence. In certain aspects, the modified endogenous OSD1-1A and modified endogenous OSD1-3A together disrupt the progression of meiosis II division in a plant cell and produce non-reduced gametes. In certain aspects, the activity of the endogenous OSD1-1A polynucleotide or polypeptide is suppressed using genome editing approaches, for example, by genetically modifying the endogenous OSD1-1A. In some approaches, the suppression results from a nucleotide modification of the endogenous maize OSD1-1A polynucleotide sequences. In some embodiments, the nucleotide modification is a deletion, addition, or substitution of one or more nucleotides. In certain embodiments, the suppression results from an amino modification of the endogenous OSD1-1A polypeptide sequence. In some embodiments, the amino acid modification is a deletion, addition, or substitution of one or more amino acids. In certain aspects, the activity of the endogenous OSD1-1A polynucleotide or polypeptide is suppressed using RNA-based silencing approaches, for example, antisense, microRNA, RNAi, or hairpin molecule, described elsewhere herein. In certain embodiments, the suppression of OSD1-1A is a knock-out of the gene.

In certain embodiments, the disclosure includes a method for obtaining a maize plant with a modified endogenous OSD1-3A polynucleotide sequence. In certain aspects, the modified endogenous OSD1-1A and modified endogenous OSD1-3A together disrupt the progression of meiosis II division in a plant cell and produce non-reduced gametes. In certain aspects, the activity of the endogenous OSD1-3A polynucleotide or polypeptide is suppressed using genome editing approaches, for example, by genetically modifying the endogenous OSD1-3A. In some approaches, the suppression results from a nucleotide modification of the endogenous maize OSD1-3A polynucleotide sequences. In some embodiments, the nucleotide modification is a deletion, addition, or substitution of one or more nucleotides. In certain embodiments, the suppression results from an amino modification of the endogenous OSD1-3A polypeptide sequences. In some embodiments, the amino acid modification is a deletion, addition, or substitution of one or more amino acids. In certain aspects, the activity of the endogenous OSD1-3A polynucleotide or polypeptide is suppressed using RNA-based silencing approaches, for example, antisense, microRNA, RNAi, or hairpin molecule, described elsewhere herein. In certain embodiments, the suppression of OSD1-3A is a knock-out of the gene. In certain embodiments, both OSD1-1A and OSD1-3A are suppressed in order to obtain disruption of the progression of meiosis II division in a maize cell.

In certain embodiments provided herein is a method of producing clonal, non-reduced, non-recombined maize seed by crossing a maize plant that comprises suppressed activity of an endogenous Spo11 polynucleotide or polypeptide, an endogenous Rec8 polynucleotide or polypeptide, an endogenous OSD1-1A polynucleotide or polypeptide, and an endogenous OSD1-3A polynucleotide or polypeptide with a male maize parent that enables the development of the clonal, non-reduced, non-recombined gamete into an embryo. A Tetraploid Haploid Inducer can be used to induce the clonal gamete to develop into an embryo and enable proper endosperm development (by maintaining the proper female: male genome ratio), which results in the production of clonal, non-reduced, non-recombined seed. As used herein, the term “Tetraploid Haploid Inducer” (THI) refers to a tetraploid version of a maize maternal haploid inducer line. A maize haploid inducer line is capable of inducing gynogenesis, resulting in the production of maternal haploid progeny that originate exclusively from the egg cell. As a tetraploid, THI can be used to induce maternal diploid progeny when used as a male in crosses with female lines capable of producing unreduced egg cells. Marker analysis of female meiotic behavior in unreduced eggs is greatly simplified by elimination of the male genome from the progeny. In certain aspects, the Tetraploid Haploid Inducer is the maize Tetraploid Haploid Inducer (w/R-navajo). The resulting seeds that are non-reduced, non-recombined, and may be grown into plants and the seed harvested from those plants.

In yet another embodiment, the present disclosure is directed to a transgenic plant or plant cells comprising the Spo11, Rec8, OSD1-1A, and/or OSD1-3A polynucleotides or suppression polynucleotides described herein or combinations thereof. The Spo11, Rec8, OSD1-1A, and/or OSD1-3A polynucleotides or suppression polynucleotides may be stably incorporated into the plant's genome or transiently expressed.

Preferred plants include but are not limited to maize, soybean, sunflower, sorghum, canola, wheat, alfalfa, cotton, rice, barley, tomato, switchgrass, miscanthus, triticale and millet. In another embodiment, the plant is a modified maize plant or maize plant cell. Another embodiment is the seed from the transgenic, modified plant. The plants of the disclosure can have altered Spo11, Rec8, OSD1-1A, OSD1-3A expression level or activity as compared to a control plant.

One embodiment of the current disclosure is a method of obtaining a maize plant that produces clonal, non-reduced, non-recombined gametes by suppressing in the maize plant the activity of an endogenous Spo11 polynucleotide or polypeptide, an endogenous Rec8 polynucleotide or polypeptide, an endogenous OSD1-1A polynucleotide or polypeptide, and an endogenous OSD1-3A polynucleotide or polypeptide. Accordingly, one embodiment is a maize plant that produces clonal, non-reduced, non-recombined gametes, where the maize plant comprises suppressed activity of an endogenous Spo11 polynucleotide or polypeptide, an endogenous Rec8 polynucleotide or polypeptide, an endogenous OSD1-1A polynucleotide or polypeptide, and an endogenous OSD1-3A polynucleotide or polypeptide. In some embodiments, the Spo11 polynucleotide is selected from the group consisting of: a polynucleotide that encodes the polypeptide of the sequence set forth in SEQ ID NO:16; a polynucleotide comprising the sequence set forth in SEQ ID NO:13, 14, 15, or 19; and a polynucleotide having at least 80% sequence identity to the sequence set forth in SEQ ID NO: 13, 14, 15, or 19 and the Spo11 polypeptide is selected from the group consisting of: a polypeptide comprising the sequence set forth in SEQ ID NO: 16; a polypeptide that is at least 80% identical to the amino acid sequence of SEQ ID NO:16; a polypeptide that is encoded by a nucleic acid molecule comprising a nucleotide sequence that is at least 80% identical to the sequence set forth in SEQ ID NO:13, 14 or 15. In some embodiments, the Rec8 polynucleotide is selected from the group consisting of: a polynucleotide that encodes the polypeptide of the sequence set forth in SEQ ID NO:12, a polynucleotide comprising the sequence set forth in SEQ ID NO:9, 10, 11 or 20; and a polynucleotide having at least 80% sequence identity to the sequence set forth in SEQ ID NO: 9, 10, 11 or 20; and the Rec8 polypeptide is selected from the group consisting of: a polypeptide comprising the sequence set forth in SEQ ID NO: 12; a polypeptide that is at least 80% identical to the amino acid sequence of SEQ ID NO:12; a polypeptide that is encoded by a nucleic acid molecule comprising a nucleotide sequence that is at least 80% identical to the sequence set forth in SEQ ID NO:9, 10 or 11. In some embodiments, the endogenous OSD1-1A polynucleotide or polypeptide is selected from the group consisting of: a polynucleotide that encodes the polypeptide of the sequence set forth in SEQ ID NO:4; a polynucleotide comprising the sequence set forth in SEQ ID NO:1, 2, 3 or 21; and a polynucleotide having at least 80% sequence identity to the sequence set forth in SEQ ID NO: 1, 2, 3, or 21; and the OSD1-1A polypeptide is selected from the group consisting of: a polypeptide comprising the sequence set forth in SEQ ID NO:4; a polypeptide that is at least 80% identical to the amino acid sequence set forth in SEQ ID NO:4; a polypeptide that is encoded by a nucleic acid molecule comprising a nucleotide sequence that is at least 80% identical to the sequence set forth in SEQ ID NO:1, 2 or 3. In some embodiments, the endogenous OSD1-3A polynucleotide is selected from the group consisting of: a polynucleotide that encodes the polypeptide of the sequence set forth in SEQ ID NO:8 a polynucleotide comprising the sequence set forth in SEQ ID NO:5, 6, 7, or 22; and a polynucleotide having at least 80% sequence identity to the sequence set forth in SEQ ID NO:5, 6, 7, or 22 and the OSD1-3A polypeptide is selected from the group consisting of: a polypeptide comprising the sequence set forth in SEQ ID NO:8; a polypeptide that is at least 80% identical to the amino acid sequence of SEQ ID NO:8; a polypeptide that is encoded by a nucleic acid molecule comprising a nucleotide sequence that is at least 80% identical to the sequence set forth in SEQ ID NO: 5, 6 or 7.

In certain embodiments, the activity of the endogenous Spo11 polynucleotide or polypeptide, Rec8 polynucleotide or polypeptide, OSD1-1A polynucleotide or polypeptide, and/or OSD1-3A polynucleotide or polypeptide, or combinations thereof is suppressed using genome editing. In certain embodiments, the suppression of the Spo11, Rec8, OSD1-1A or OSD1-3A gene is a knock-out of the gene. In some approaches, the suppression results from a nucleotide modification of the endogenous Spo11, Rec8, OSD1-1A or OSD1-3A polynucleotide sequences. In some embodiments, the nucleotide modification is a deletion, addition, or substitution of one or more nucleotides. In certain embodiments, the suppression results from an amino modification of the endogenous Spo11, Rec8, OSD1-1A or OSD1-3A polypeptide sequence. In some embodiments, the amino acid modification is a deletion, addition, or substitution of one or more amino acids. In certain embodiments, the activity of the endogenous Spo11, Rec8, OSD1-1A or OSD1-3A polynucleotide or polypeptide is suppressed using RNA-based silencing approaches, for example, antisense, microRNA, RNAi, or hairpin molecule, described elsewhere herein.

In certain embodiments, the gene editing approach and anti-sense, cosuppression, viral-suppression, hairpin suppression, stem-loop suppression, RNAi-based approaches, and small RNA-based approaches are used alternatively or in addition to to suppress activity of the endogenous Spo11, Rec8, OSD1-1A or OSD1-3A polynucleotide or polypeptides, thereby producing non-reduced, non-recombined and/or clonal gametes or combinations thereof. As one example, the suppression of both OSD1-1A and OSD1-3A in maize results in the disruption of meiosis and non-reduction of gametes. In another example, the suppression of both Spo11 and Rec8 in maize results in non-recombined gametes. With suppression of all four (Spo11, Rec8, OSD1-1A and OSD1-3A) in maize, non-reduced, non-recombined, clonal gametes may be produced. In some examples, the suppressed Spo11, Rec8, OSD1-1A or OSD1-3A polynucleotides or polypeptides are in a maize plant or plant cell.

In certain embodiments, the methods and compositions include a plant cell that has the modified endogenous Spo11, Rec8, OSD1-1A and/or OSD1-3A gene and/or suppression polynucleotide targeting Spo11, Rec8, OSD1-1A and/or OSD1-3A or combinations thereof. The plant may produce non-reduced, non-recombined and/or clonal female and/or male gametes. In other embodiments, the plant cell having the modified endogenous Spo11, Rec8, OSD1-1A and/or OSD1-3A gene or suppression polynucleotides targeting Spo11, Rec8, OSD1-1A and/or OSD1-3A or combinations thereof may be combined with one or more genes involved in recombination that have been modified, for example, by gene-editing technologies, to have decreased recombination activity in order to create plants that produce non-reduced, or non-reduced and non-recombined, female and/or male gametes. Additionally, the modified Spo11, Rec8, OSD1-1A and/or OSD1-3A gene and/or Spo11, Rec8, OSD1-1A and/or OSD1-3A suppression polynucleotides can be combined with other genes that are modified and/or other suppression polynucleotides that target other genes of interest. For example, the plant cell having the modified endogenous Spo11, Rec8, OSD1-1A or OSD1-3A gene or suppression polynucleotides targeting Spo11, Rec8, OSD1-1A or OSD1-3A may be combined or stacked with a suppression polynucleotide that targets genes that play a role in recombination in order to create plants that produce male and/or female gametes that are clonal, non-reduced and non-recombined or combinations thereof. For example, the recombination target genes include but are not limited to PRD1 (De Must et al, (2007) EMBO J. 26:4126-4137), TAM, TDM, Ago 104 (Singh et al, (2011) Plant Cell. 23:443-458), AMI, AM2, PAM1, PAM2, AS1, DSY1, DY1, ST1, ELI, DV1, VA1, VA2, POI, NRF4, and the like and combinations thereof.

Plant cells from the non-reduced, non-recombined and/or clonal female or male gametes can be stacked with traits desirable for disease or herbicide resistance (e.g., fumonisin detoxification genes (U. S. Pat. No. 5,792,931); avirulence and disease resistance genes (Jones et al, (1994) Science 266:789-793; Martin et al, (1993) Science 262: 1432-1436; Mindrinos et al, (1994) Cell 78: 1089-1099); acetolactate synthase (ALS) mutants that lead to herbicide resistance, such as the S4 and/or Hra mutations; inhibitors of glutamine synthase, such as phosphinothricin or basta (e.g., bar gene); and glyphosate resistance (EPSPS gene); traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al, (1988) J. Bacteriol. 170:5837-5847) facilitate expression of polyhydroxyalkanoates (PHAs)), the disclosures of which are herein incorporated by reference. One could also combine various polynucleotides, for example, polynucleotides providing agronomic traits such as male sterility (e.g., see U.S. Pat. No. 5,583,210), stalk strength, flowering time, or transformation technology traits, such as cell cycle regulation or gene targeting (e.g., WO 99/61619, WO 00/17364, and WO 99/25821), the disclosures of which are herein incorporated by reference.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, W099/25821, W099/25854, WO99/25840, W099/25855, and W099/25853, all of which are herein incorporated by reference.

In certain embodiments, methods are provided herein for producing clonal, non-recombined, and non-reduced male and/or female gametes or combinations thereof as well as and methods of maintaining clonality in offspring. Clonality may be determined using any suitable technique, for example, comparing the SNP pattern of the hybrid progenitor plants with progeny plants using heterozygous SNP markers. In some embodiments, the method comprises introducing into a plant a modification, for example, a suppression polynucleotide that suppresses the activity of the endogenous Spo11, Rec8, OSD1-1A and OSD1-3A polynucleotides or polypeptides, or combinations thereof, thereby producing clonal, non-reduced, and/or non-reduced male or female gametes or combinations thereof. In certain embodiments, the method comprises modifying an endogenous Spo11, Rec8, OSD1-1A and/or OSD1-3A target polynucleotides, for example, Spo11, Rec8, OSD1-1A and/or OSD1-3A genes, using gene editing approaches.

A further embodiment includes methods of maintaining clonality in a progeny plant that includes regenerating a progeny plant from a parent plant that has non-reduced, non-recombined clonal female or male gametes and has the same genotype as the parent plant. In some examples, the endogenous Spo11, Rec8, OSD1-1A and OSD1-3A gene is suppressed in the parent and progeny plant. In an aspect, the methods disclosed herein can further comprise the step of introducing into the plant genome a suppression of the endogenous Spo11, Rec8, OSD1-1A and OSD1-3A gene or combinations thereof, and regenerating a plant having such an altered genome.

In certain embodiments, the maize plant is a hybrid. In certain embodiments, the maize plant is an inbred. In certain embodiments, the maize plant is a female or male parent. Gametes obtained from this plant may be clonal, non-reduced, non-recombined gametes. The gametes may be male or female gametes or combinations thereof.

One embodiment of the disclosure includes a method of obtaining a maize plant that produces clonal, non-reduced, non-recombined male and/or female gametes. In certain embodiments, maize plants that have suppressed activity with respect to its endogenous Spo11, Rec8, OSD1-1A, and OSD1-3A polynucleotides and polypeptides are either heterozygous or homozygous for the suppression. These plants may be crossed, intercrossed, or selfed until a maize plant is obtained that comprises suppressed activity for each of these endogenous genes: Spo11, Rec8, OSD1-1A and OSD1-3A activity. In certain embodiments, Spo11 and Rec8 heterozygotes are intercrossed to generate homozygous knockouts of Spo11 and Rec8 since homozygote knock-outs of Spo11 and Rec8 in maize are individually male and female sterile and cannot be intercrossed.

In some embodiments, the homozygous suppression is caused by knocking out the gene. In certain embodiments, the maize plant is a hybrid. In certain embodiments, the maize plant is an inbred. In certain embodiments, the maize plant is a female or male parent. Gametes obtained from this plant may be clonal, non-reduced, non-recombined male and/or female gametes. Using methods described herein, seed produced from this plant may produce progeny that are non-reduced, non-recombined and clonal with respect to the parent plant, for example, when combined with haploid induction, parthenogenic or genome elimination approaches.

In certain embodiments, a method of producing a plant that comprises clonal, non-reduced and non-recombined female or male gametes or combinations thereof as compared with the parent plant comprises intercrossing or selfing a maize plant that has suppressed activity with respect to its endogenous Spo11, Rec8, OSD1-1A, and OSD1-3A polynucleotides and polypeptides. The resulting maize plant may be heterozygous, homozygous or a combination thereof for suppression of Spo11, Rec8, OSD1-1A, and OSD1-3A activity. For example, a maize plant is obtained from this method may be homozygous for the suppressed endogenous Spo11, Rec8, OSD1-1A and OSD1-3A activity. In certain embodiments, the suppression may be heterozygous, homozygous or a combination thereof. In some embodiments, the homozygous suppression is caused by knocking out the gene. In certain embodiments, the maize plant is a hybrid. In certain embodiments, the maize plant is an inbred. In certain embodiments, the maize plant is a female or male parent. Gametes obtained from this plant may be clonal, non-reduced, non-recombined male or female gametes.

The female parent that has suppressed Spo11, Rec8, and OSD1-1A, and OSD1-3A activity may be crossed with a tetraploid haploid inducer (THI) serving as a male parent to produce seed that is non-reduced and non-recombined. With respect to maize, the non-reduced and non-recombined seed may be diploid “haploids” (2n chromosome number) following pollination with the THI and have chromosomes only from the female parent. This will allow for clonal reproduction of the parent plant through seeds.

To facilitate with the identification of non-reduced and non-recombined seed, in certain embodiments, the tetraploid haploid inducer (THI) male parent comprises w/R-nj. See, WO2016179522 published application, herein incorporated by reference in its entirety. The use of w/R-nj in this method allows for the visual selection of non-reduced and non-recombined progeny since the haploid kernels with the female parent chromosomes will have a dark endosperm and normal (light-colored) embryo relative to regular seed produced from male and female parents which will have both dark endosperm and dark embryo. In this manner, 2n ‘haploids” may be identified by selecting seed with a colored endosperm cap and a colorless embryo. The other seeds (non-haploid seeds) may be tetraploids produced following pollination with a THI.

The seeds may be further evaluated, for example, by growing a plant from the non-reduced and non-recombined seed to determine whether the plant resembles and has the phenotype of the female parent plant, for example, when the plant is maize, whether the plant has similarities in plant height, maturity, flag leaf, and/or tassel phenotype. Alternatively or in addition, the heterozygous SNP pattern can be determined and compared to the SNP pattern of the hybrid progenitor plants. The SNP pattern from the parent and from the non-reduced and non-recombined progeny plants should share the same SNP pattern, indicating that no recombination had occurred in the progeny plants. The plants may be evaluated for ploidy status, for example, diploid, haploid or tetraploid status, to determine whether a reduction division in meiosis occurred. In certain instances, progeny plants can exhibit male sterility and/or reduced female fertility.

In certain embodiments, a parent plant homozygous for Spo11, Rec8, OSD1-1A, and OSD1-3A suppression comprises a haploid inducer gene so that non-recombined, non-reduced clonal progeny plants may be self-generated. Examples of haploid inducer genes include but are not limited to Matrilineal (MTL) gene. See, for example, Nature. 542:105-109 (2017), herein incorporated by reference in its entirety.

In certain embodiments, methods are provided for producing a plant that produces clonal, non-reduced, non-recombined seeds. For example, the female gametophyte or gamete produced using the methods and compositions described herein may undergo genome elimination to produce clonal, non-reduced, non-recombined maize seed using any suitable genome elimination approach. In certain embodiments, the method includes crossing a maize plant suppressed for the activity of an endogenous Spo11 polynucleotide or polypeptide, an endogenous Rec8 polynucleotide or polypeptide, an endogenous OSD1-1A polynucleotide or polypeptide, and an endogenous OSD1-3A polynucleotide or polypeptide with a maize male plant expressing a modified CENH3. Plants with modified CENH3 genes and polypeptides are described in U.S. Pat. Nos. 9,215,849 and 8,618,354, herein incorporated by reference in their entirety. In certain embodiments, the modified CENH3 allows for the elimination of the male or female parent genome in the zygote resulting from the cross of these two plants, such that the resulting progeny is non-recombined, non-reduced and clonal of one of the parent plants. In this manner, the seed obtained from a plant produced from this method allow for the clonal reproduction of the maize plant through seed.

In certain embodiments, methods are provided for producing a plant that produces clonal, non-reduced, non-recombined seeds, where the seeds comprises parthenogenetically-derived clonal embryos. For example, the female gametophyte or gamete produced using the methods and compositions described herein may undergo parthenogenesis to produce clonal, non-reduced, non-recombined maize seed using any suitable parthenogenic approach. In certain embodiments, the method includes crossing a maize plant suppressed for the activity of an endogenous Spo11 polynucleotide or polypeptide, an endogenous Rec8 polynucleotide or polypeptide, an endogenous OSD1-1A polynucleotide or polypeptide, and an endogenous OSD1-3A polynucleotide or polypeptide with a maize male plant expressing with a male parent that comprises a gene that induces a gamete to divide as an embryo. Exemplary genes that induce a gamete to divide as an embryo include but are not limited to BBML genes. See, for example, the ASGR-BBML gene in US Patent Application Publication No. 20160304901, incorporated herein by reference in its entirety. The resulting seeds obtained from this cross are clonal, non-reduced, non-recombined seeds. Plants produced from this method allow for the clonal reproduction of the maize plant through seed. In the methods described herein, the parent plant may be a hybrid plant.

Various types of promoters can be employed in the methods and compositions provided herein. Promoters can drive expression in a manner that is cell-type-preferred, cell-type-specific, tissue-preferred or tissue-specific. Examples of promoters under developmental control include promoters that preferentially initiate transcription in certain tissues, such as leaves, roots, seeds or ovules. Such promoters are referred to as “tissue preferred”. Promoters which initiate transcription only in certain tissue are referred to as “tissue specific”. A “cell type” preferred promoter primarily drives expression in certain cell types in one or more organs, for example, vascular cells in roots, leaves or ovules. An “inducible” or “repressible” promoter is a promoter which is under environmental or chemical control. Examples of environmental conditions that may affect transcription by inducible promoters include anaerobic conditions or the presence of light. Tissue specific, tissue preferred, cell type specific, cell type preferred and inducible promoters constitute the class of “non-constitutive” promoters. A “constitutive” promoter is a promoter that is active under most environmental conditions and in all tissues throughout development.

Non-limiting examples of constitutive promoters include, for example, the core promoter of the Rsyn7 promoter and other constitutive promoters disclosed in WO 1999/43838 and U.S. Pat. No. 6,072,050; the core CaMV 35S promoter (Odell et al, (1985) Nature 313:810-812); the rice actin promoter (McElroy et al., (1990) Plant Cell 2: 163-171); the ubiquitin promoter (Christensen et al, (1989) Plant Mol. Biol. 12:619-632 and Christensen et al, (1992) Plant Mol. Biol. 18:675-689); the pEMU promoter (Last et al, (1991) Theor. Appl. Genet. 81:581-588); the MAS promoter (Velten et al, (1984) EMBO J. 3:2723-2730); the ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

One of skill will recognize that the sequences encoding elements or polypeptides can be placed into an expression cassette or constructs. Expression cassettes or constructs are discussed elsewhere herein. Any promoter of interest can be operably linked to the sequence encoding the elements or polypeptides, including, for example, constitutive promoters, tissue-preferred promoters, tissue-specific promoters, including promoters that are functional during meiosis. Such promoters have been described elsewhere herein or are known in the art.

By “promoter” is intended a regulatory region of DNA usually comprising a TATA box capable of directing RNA polymerase II to initiate RNA synthesis at the appropriate transcription initiation site for a particular polynucleotide sequence. A promoter may additionally comprise other recognition sequences generally positioned upstream or 5′ to the TATA box, referred to as upstream promoter elements, which influence the transcription initiation rate. The promoter sequences disclosed herein regulate (i.e., activate) transcription from the promoter region.

In some embodiments, the methods and compositions include isolated polynucleotides comprising Spo11, Rec8, OSD1-1A, and/or OSD1-3A upstream region sequences. The Spo11, Rec8, OSD1-1A, and/or OSD1-3A upstream region nucleotide sequences include those set forth in SEQ ID NOS: 19, 20, 21 and 22, active variants and fragments thereof. In one embodiment, an expression construct includes any of the polynucleotides set forth in SEQ ID NOS: 19, 20, 21 and 22 operably linked to the polynucleotide of interest or any polynucleotide having at least 95%, 96%, 97%, 98% or 99% sequence identity to the sequence set forth in SEQ ID NO:19, 20, 21 and 22, wherein said polynucleotide retains the ability to direct expression of an operably linked polynucleotide in prior to or during meiosis.

Fragments and variants of each of the upstream region nucleotide sequences set forth in SEQ ID NOS:19, 20, 21 and 22 are further provided herein. Fragments of a upstream region polynucleotide may retain biological activity and, hence, retain transcriptional regulatory activity. Thus, fragments of a upstream region nucleotide sequence may range from at least about 20 nucleotides, about 50 nucleotides, about 100 nucleotides, and up to the full-length polynucleotide of the disclosure. Polynucleotides that are fragments of the upstream region polynucleotides comprise at least 16, 20, 50, 75, 100, 150, 200, 250, 300, 350, 400, 450, 500, 550, or 600, nucleotides.

For an upstream region polynucleotide, a variant comprises a deletion and/or addition of one or more nucleotides at one or more internal sites within the native polynucleotide and/or a substitution of one or more nucleotides at one or more sites in the native polynucleotide. Generally, variants of a particular upstream region polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Thus, any of the Spo11, Rec8, OSD1-1A, and/or OSD1-3A upstream region sequences, variants, and fragments thereof may be utilized to regulate a number of genes and developmental processes prior to or during meiosis. The Spo11, Rec8, OSD1-1A, and/or OSD1-3A promoter may be used to ectopically express an RNA resulting in protein production, silencing, gene modification, modification of other RNA(s), or catalysis of a reaction.

As discussed herein, various promoters can be employed in the methods and compositions provided herein, including: promoters to express sequences encoding suppression polynucleotides that suppress Spo11, Rec8, OSD1-1A, and/or OSD1-3A activity, for example, maize ubiquitin promoter, soy ubiquitin promoter, any of the upstream region sequences described herein. A Spo11, Rec8, OSD1-1A, and/or OSD1-3A upstream region sequence, variant, or fragment thereof can be operably linked to any of the sequences encoding suppression polynucleotides or polypeptides disclosed herein or known in the art. In one embodiment, the Spo11, Rec8, OSD1-1A, and/or OSD1-3A upstream region may be utilized in constructs designed to modify or alter the meiotic process. In such an embodiment, the construct may be used to express suppression polynucleotides that target LAM, OSD1, SPOII, TDM, PRD1, PRD2, PRD3, DFOI, REC8, AMI, AM2, PAM1, PAM2, AS1, DSY1, DY1, ST1, ELI, DV1, VA1, VA2 and/or POL.

It is recognized that additional domains can be added to the upstream region sequences disclosed herein and thereby modulate the level of expression, the developmental timing of expression, or the tissue type that expression occurs in. See, particularly, Australian Patent Number AU-A-77751/94 and U.S. Pat. Nos. 5,466,785 and 5,635,618.

Any of the promoter sequences employed herein can be modified to provide for a range of expression levels of the heterologous nucleotide sequence. Thus, less than the entire promoter region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of promoter deletions if, for example, there is a negative regulatory element (for a repressor) that is removed during the truncation process. Generally, at least about 20 nucleotides of an isolated promoter sequence will be used to drive expression of a nucleotide sequence.

Methods are available in the art for determining if a promoter sequence retains the ability to regulate transcription in the desired temporal and spatial pattern. Such activity can be measured by Northern blot analysis. See, for example, Sambrook et al, (1989) Molecular Cloning: A Laboratory Manual (2d ed., Cold Spring Harbor Laboratory Press, Plainview, New York), herein incorporated by reference. Alternatively, biological activity of the promoter can be measured using assays specifically designed for measuring the activity and/or level of the polypeptide being expressed from the promoter. Such assays are known in the art.

It is recognized that to increase transcription levels, enhancers may be utilized in combination with the promoter disclosed herein. Enhancers are nucleotide sequences that act to increase the expression of a promoter region. Enhancers are known in the art and include the SV40 enhancer region, the 35 S enhancer element, and the like. Some enhancers are also known to alter normal promoter expression patterns, for example, by causing a promoter to be expressed constitutively when without the enhancer, the same promoter is expressed only in one specific tissue or a few specific tissues.

Modifications of the promoters or upstream region sequences disclosed herein can provide for a range of expression of the heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level or at about 1/10 transcripts to about 1/100 transcripts to about 1/1,000 transcripts.

The polynucleotides comprising the Spo11, Rec8, OSD1-1A, and/or OSD1-3A upstream region sequences disclosed herein, see, for example, SEQ ID NOS:19, 20, 21,or 22, as well as variants and fragments thereof, are useful in the genetic manipulation of any host cell, preferably plant cell.

The Spo11, Rec8, OSD1-1A, and/or OSD1-3A upstream region sequences disclosed herein, see, for example, SEQ ID NOS: 19, 20, 21, or 22, as well as variants and fragments thereof may be operably linked to a heterologous polynucleotide of interest. In this manner, the Spo11, Rec8, OSD1-1A, and/or OSD1-3A promoter polynucleotides of the disclosure are provided in expression cassettes along with a polynucleotide sequence of interest for expression in the host cell of interest. The Spo11, Rec8, OSD1-1A, and/or OSD1-3A promoter sequences of the disclosure may be useful in regulating the temporal and/or the spatial expression of polynucleotides of interest during meiosis.

As disclosed elsewhere herein, the Spo11, Rec8, OSD1-1A, and/or OSD1-3A upstream region sequences may be targets for Spo11, Rec8, OSD1-1A, and/or OSD1-3A suppression polynucleotides, including but not limited to promoter-inverted repeats or hairpinRNA.

Methods are also provided for the use of the Spo11, Rec8, OSD1-1A, and OSD1-3A polynucleotide and polypeptide sequences disclosed herein to affect meiosis. In certain embodiments, the method comprises increasing the activity or level of the Spo11, Rec8, OSD1-1A, and OSD1-3A polynucleotide and polypeptide sequences in a plant or plant part.

Numerous methods for introducing foreign genes into plants are known and can be used to insert a Spo11, Rec8, OSD1-1A, and/or OSD1-3A polynucleotide into a plant host, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The introduction of DNA constructs, for example, recombinant or suppression DNA constructs, of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods. eds. Gamborg and Phillips. Springer-Verlag Berlin Heidelberg New York, 1995; U.S. Pat. No. 5,736,369 (meristem) 185), all of which are herein incorporated by reference. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Once transformed, these cells can be used to regenerate transgenic plants. Examples of such methods for regenerating plant tissue are disclosed in Shahin, (1985) Theor. Appl. Genet. 69:235-40; U.S. Pat. No. 4,658,082; Simpson, et al., supra and US Patent Application Ser. Numbers 913,913 and 913,914, both filed Oct. 1, 1986, as referenced in U.S. Pat. No. 5,262,306, issued Nov. 16, 1993, the entire disclosures therein incorporated herein by reference.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims. This disclosure can be better understood by reference to the following non-limiting examples. It will be appreciated by those skilled in the art that other embodiments of the disclosure may be practiced without departing from the spirit and the scope of the disclosure as herein disclosed and claimed.

### Example 1: Plant Materials of Mime (Mitosis Instead of Meiosis) Parent Plants

MiMe parent plants were a mixture of Zmspo11/Zmrec8 double homozygous TUSC insertion mutants (Golubovskaya I, Hamant O, Timofejeva L, Wang C R, Braun D, Meeley R, Cande W Z (2006) Alleles of afd1 dissect REC8 functions during meiotic prophase I. Journal of Cell Science 119: 3306-3315) and a transgenic line containing two artificial miRNA's (amiRNA) targeting ZmOSD1-1A and ZmOSD1-3A. Incompletely penetrant transgenic amiRNA lines silencing ZMOSD1-1A and ZmOSD1-3A that gave both reduced and non-reduced male and female gametes were selected for crossing with Zmspo11/Zmrec8 double heterozygous plants. These plants were self-pollinated, and progeny were analyzed with gene specific markers to determine zygosity of Zmspo11, Zmrec8 and ZMOSD1-1A/ZMOSD1-3A amiRNAs. Plants homozygous for Zmspo11 and Zmrec8 and hemi- or homo-zygous for ZMOSD1A-1/ZMOSD1-3A amiRNAs were selected (frequency 1/32-1/64).

TABLE 2
SNP marker analysis of progeny from
three hybrid MiMe female parents

| MiMe Hybrid | | |
|---|---|---|
| Parent Plant | Progeny | SNP marker analysis |
| Hybrid | 2 - 2n “haploid” | 119 heterozygous SNP |
| female #1 | progeny | markers tested across all |
| | | 10 chromosomes. Each |
| | | with same SNP marker |
| | | profile as Hybrid Female #1 |
| Hybrid | 4 - 2n “haploid” | 114 heterozygous SNP |
| female #2 | progeny | markers tested across all |
| | | 10 chromosomes. Each |
| | | with same SNP marker |
| | | profile as Hybrid Female #2 |
| Hybrid | 4 - 2n “haploid” | 128 heterozygous SNP |
| female #3 | progeny | markers tested across all |
| | | 10 chromosomes. Each |
| | | with same SNP marker |
| | | profile as Hybrid Female #3. |

1-32. (canceled)

33. A method for obtaining a maize plant cell with a modified endogenous Rec8 polynucleotide sequence, the method comprising:
a) introducing a genetic modification into an endogenous Rec8 polynucleotide sequence in a maize plant cell, wherein the genetic modification disrupts the orientation of kinetochores and subsequent random distribution of chromatids during meiosis II in the maize plant cell, wherein the endogenous Rec8 polynucleotide comprises:
i. a polynucleotide that encodes the polypeptide of SEQ ID NO:12,
ii. a polynucleotide comprising the sequence set forth in SEQ ID NO:9, 10, 11 or 20; or
iii. a polynucleotide having at least 80% sequence identity to SEQ ID NO: 9, 10, 11 or 20; and
a) obtaining a maize plant cell having the modified endogenous Rec8 polynucleotide sequence.

34. The method of claim 33, wherein the genetic modification is a deletion, addition, or substitution of one or more nucleotides.

35. The method of claim 33, wherein the genetic modification is introduced by a nuclease selected from the group consisting of: a TALEN, a meganuclease, a zinc finger nuclease, and a CRISPR-associated nuclease.

36. The method of claim 35, wherein the genetic modification is introduced by a Cas9 endonuclease guided by at least one guide RNA.

37. The method of claim 33, further comprising obtaining a maize plant from the maize plant cell.

38. The method of claim 37, wherein the maize plant is a hybrid or inbred.

39. The method of claim 37, wherein the introduced genetic modification suppresses activity of the endogenous Rec8 polynucleotide or polypeptide in the maize plant.

40. The method of claim 33, wherein the method further comprises:
suppressing in the maize plant cell the activity of an endogenous Spo11 polynucleotide or polypeptide, wherein the Spo11 polynucleotide comprises:
i. a polynucleotide that encodes the polypeptide of SEQ ID NO:16;
ii. a polynucleotide comprising the sequence set forth in SEQ ID NO:13, 14, 15, or 19; or
iii. a polynucleotide having at least 80% sequence identity to SEQ ID NO: 13, 14, 15, or 19;
wherein the Spo11 polypeptide comprises:
i. a polypeptide comprising SEQ ID NO: 16;
ii. a polypeptide that is at least 80% identical to the amino acid sequence of SEQ ID NO:16; or
iii. a polypeptide that is encoded by a nucleic acid molecule comprising a nucleotide sequence that is at least 80% identical to SEQ ID NO:13, 14 or 15;

41. The method of claim 40, comprising suppressing in the maize plant cell activity of an endogenous Spo11 polynucleotide or polypeptide by introducing a nucleotide modification into its polynucleotide sequence or an amino modification into its polypeptide sequence.

42. The method of claim 41, wherein the nucleotide modification is a deletion, addition, or substitution of one or more nucleotides.

43. The method of claim 41, wherein the amino acid modification is a deletion, addition, or substitution of one or more amino acids.

44. The method of claim 42, wherein said the nucleotide modification is introduced by a nuclease selected from the group consisting of: a TALEN, a meganuclease, a zinc finger nuclease, and a CRISPR-associated nuclease.

45. The method of claim 44, wherein the nucleotide modification is introduced by a Cas9 endonuclease guided by at least one guide RNA.

46. The method of claim 41, further comprising obtaining a maize plant from the maize plant cell having introduced modifications in Rec8 and Spo11.

47. The method of claim 46, crossing maize plants that are heterozygous for suppressed endogenous Rec8 activity with maize plants that are heterozygous for suppressed endogenous Spo11 activity until a maize plant is obtained that is homozygous for knockouts of endogenous Rec8 and Spo11 polynucleotides.

48. The method of claim 47, further comprising obtaining non-recombined gametes from the maize plant homozygous for the suppressed endogenous Rec8 and Spo11 activity.

49. A maize plant comprising a modified endogenous Rec8 polynucleotide sequence, wherein the maize plant comprises an introduced genetic modification into an endogenous Rec8 polynucleotide sequence, wherein the endogenous Rec8 polynucleotide comprises:
i. a polynucleotide that encodes the polypeptide of SEQ ID NO:12,
ii. a polynucleotide comprising the sequence set forth in SEQ ID NO:9, 10, 11 or 20; or
iii. a polynucleotide having at least 80% sequence identity to SEQ ID NO: 9, 10, 11 or 20.

50. The maize plant of claim 49, wherein the maize plant is a hybrid or inbred.

51. The maize plant of claim 49, wherein the activity of the endogenous Rec8 polynucleotide or polypeptide is suppressed by introducing a nucleotide modification into its polynucleotide sequence or an amino modification into its polypeptide sequence.

52. The maize plant of claim 51, wherein the nucleotide modification is a deletion, addition, or substitution of one or more nucleotides.

53. The maize plant of claim 51, wherein the amino acid modification is a deletion, addition, or substitution of one or more amino acids.

54. The maize plant of claim 53, wherein said the nucleotide modification is introduced by a nuclease selected from the group consisting of: a TALEN, a meganuclease, a zinc finger nuclease, and a CRISPR-associated nuclease.

55. The maize plant of claim 54, wherein the nucleotide modification is introduced by a Cas9 endonuclease guided by at least one guide RNA.

56. The maize plant of claim 49, wherein the maize plant further comprises:
suppressed activity of an endogenous Spo11 polynucleotide or polypeptide.

57. The maize plant of claim 56, wherein the Spo11 polynucleotide comprises:
iv. a polynucleotide that encodes the polypeptide of SEQ ID NO:16;
v. a polynucleotide comprising the sequence set forth in SEQ ID NO:13, 14, 15, or 19; and
vi. a polynucleotide having at least 80% sequence identity to SEQ ID NO: 13, 14, 15, or 19;
wherein the Spo11 polypeptide comprises:
iv. a polypeptide comprising SEQ ID NO: 16;
v. a polypeptide that is at least 80% identical to the amino acid sequence of SEQ ID NO:16; or
vi. a polypeptide that is encoded by a nucleic acid molecule comprising a nucleotide sequence that is at least 80% identical to SEQ ID NO:13, 14 or 15.

58. The maize plant of claim 57, wherein the activity of the endogenous Spo11 polynucleotide or polypeptide is suppressed by an introduced nucleotide modification into its polynucleotide sequence or an amino modification into its polypeptide sequence.

59. The maize plant of claim 58, wherein the nucleotide modification is a deletion, addition, or substitution of one or more nucleotides.

60. The maize plant of claim 58, wherein the amino acid modification is a deletion, addition, or substitution of one or more amino acids.

61. The maize plant of claim 59, wherein said the nucleotide modification is introduced by a nuclease selected from the group consisting of: a TALEN, a meganuclease, a zinc finger nuclease, and a CRISPR-associated nuclease.

62. The maize plant of claim 61, wherein the nucleotide modification is introduced by a Cas9 endonuclease guided by at least one guide RNA.

63. The maize plant of claim 56, wherein the maize plant is heterozygous for suppressed endogenous Rec8 activity and heterozygous for suppressed endogenous Spo11 activity.

64. The maize plant of claim 56, wherein the maize plant is homozygous for knockouts of endogenous Rec8 and Spo11 polynucleotides to produce non-recombined gametes.

65. Non-recombined gametes from the maize plant of claim 64.


SEQUENCE LISTING (22 sequences)

SEQ ID NO: 1 - DNA, 3104 bp, Zea mays
  source 1..3104: mol_type = genomic DNA
caacaaggacagagcagaaacttagccagagattcgtccaggataggggctgtatcgtagggagtagtcgttgcggagattgggtcgaagcggactggacaggagccgagcaaactgtgagtattgtattttctcttttgcggtttggagggtttggatgtggttgtttggttgcagttttccgagcgttttagtgatgtggtttttcctcttgccatgtcctttcagatctgcagaagaggttaaagggttgccgtgcggactccggagctgtgagaatctaccccgaggtgagaatctctaaacggttgtggccttgagggatgcggttagttcttggtccgattatgttagttcgcccccgaattgctttcttcagacgtgtttcgtggcttctttatgtgtaaatcccttggagggtagagcattgaggcggggttatgagtagaaccgtcgcagattaaatgattatgaccgtgtttctttacgcaatttcttcgtgctattggtacttatacactgctctcctctgcgttgcgtctttcttgcaggtaagctcttagagtcgtagtcaaagaggagaagccaggccgggaacaaacagaaaagtgaggagatttgttggtgcattagctactcacgcgtcccctgaaattctgcttgttaactccatcctaaaatttattgtgtggacccaggagaagagcaacaggagtacaggactcagtgcaccaagaataggaaagagcaaagaccataacagttgtatgagatactgtgatttctgggttcatttgtttatctgattatgcgttctttggtctcttgcatcactcatgtggttgtatgcactcctccaggaacttcaagatgcttgaagtgaggactgcaagaaggccggctctcgccgacatctctggtggtgggttctttatgaggacagtagaatcgccaggagctgtgctggtgaatggtgctgtcaagcggccggctcggcagtttctatcaccttcaagcaacaaggagaatgtgccaccagtgggagctttcagggctacaccaaagaggaggacccccttgcctgactggtacccgaggacaccactccgtgacatcacatcaatcgtcaaggtggtggactatttccttgctattgtctaattccattttgttgtatttggtgcatgcactgtactctataatatgctacagtgtggtgcaatgtttcatttgtgagtccttgtgttcagaatgcctggttagtggccaacaaagataggttatgtagtaggtttggtaattggtattcagaatgttgagccttgtacactattattctataatacaatagatctgccagttctgctagctgccttgatgacatagaatgagaaatatatacagttggattttatcattagtttagcaactcatagcaaaagattcttataatttcaaataacatagtaataatactcttacaagacattaccaacactcttttattctatcttacttgcatctagctgtgttcactattcagttttaggtgcctgaccatttaaatgtgcttaaatttgcagaaaggtcagtagtcaataaggcatgttaaatctaaatttattcttctccattgtacttaatgttaccccatttccccttattctctttattctctttgaatatttaaccgatcataatagatgttcctttgtgtcgtgactgaattactgtacttgtttgtagaaactacgcactgcaactaaaaagtcacacactttaggctgtcatgttaagaagttatatacaggattgtgtttagaaattcattgacaatttgccatgatccgtaaacttaagtcagttgttgaggatcttatttgcaacggcatgtcttcttagttgttctgaaatgtccattcatttaaacgtagcctactaaaaatgagattcctataaagattcatagtatagggtttcgacttcatctttagttttaagctttatatgaccaatctatttaggatatttgcaagaatgtcatttggtgtttttaatgattttaatcgtcttcataacttatattacagaaacatttttatgttgttacttatatacacggttacagactacacgagttatatttgttgatattggaagcgttgaaactactgatagctgatactccctccgttcgtttttatttgttgttattgacctataatgtgcttactttgtctgttacttttaattattggcagaaggtatattcttagatgttagatgtgtatagtgtttccctggaaatttgctgatgtactaactgctagctgcaggcaattgagaggaggagaagtcgtctgcagaatgctgcagctcagcagcagatccagtggacagaagacccttcccgatctgtggatccaataactccagtacaggcagagcagggtggtgtgccaacaactgtggatggtcaaggtgttggaagccctgcaacctgtttggaggatggcaagctgaagacatcgtcctatccatcatctgactgctccttgcaggccactccatccaaaccaaacgatccagctctcgcagatctcgtggagaagaagctgtccagctcgatagagcagatcgagaagatggtgcggcgaaacctgaagagaacttcgaaggccgctcagccttccaagaggaccatccagaggcgcgtcctgatgtccatgcgatgagctgagaaagctatctgctctgccatggcttcccttgtctgctgctttcttagcagcaagtcgtaaccaaccattacgccagttttgcctgaaggaggggtggggccgtgggggctcctggttctctgcttgaggaccccggggtatgcggcttactactagctctctgcttgagggtacacattatggtgactgcgtatttttaatttgagaaattagtagttctatgtatagttttatagtgtggaacggatatgagaaattagtagcaattgtgacttcgacagcttgaatactgttgttatggagtagttcctctatactgttgtttccgagtaacataacacagttttggccatcagtaatgga

SEQ ID NO: 2 - DNA, 1526 bp, Zea mays
  source 1..1526: mol_type = genomic DNA
caacaaggacagagcagaaacttagccagagattcgtccaggataggggctgtatcgtagggagtagtcgttgcggagattgggtcgaagcggactggacaggagccgagcaaactatctgcagaagaggttaaagggttgccgtgcggactccggagctgtgagaatctaccccgaggtaagctcttagagtcgtagtcaaagaggagaagccaggccgggaacaaacagaaaagtgaggagatttgttggtgcattagctactcacgcgtcccctgaaattctgcttgttaactccatcctaaaatttattgtgtggacccaggagaagagcaacaggagtacaggactcagtgcaccaagaataggaaagagcaaagaccataacagttgtatgagatactgtgatttctgggttcatttgtttatctgattatgcgttctttggtctcttgcatcactcatgtggttgtatgcactcctccaggaacttcaagatgcttgaagtgaggactgcaagaaggccggctctcgccgacatctctggtggtgggttctttatgaggacagtagaatcgccaggagctgtgctggtgaatggtgctgtcaagcggccggctcggcagtttctatcaccttcaagcaacaaggagaatgtgccaccagtgggagctttcagggctacaccaaagaggaggacccccttgcctgactggtacccgaggacaccactccgtgacatcacatcaatcgtcaaggcaattgagaggaggagaagtcgtctgcagaatgctgcagctcagcagcagatccagtggacagaagacccttcccgatctgtggatccaataactccagtacaggcagagcagggtggtgtgccaacaactgtggatggtcaaggtgttggaagccctgcaacctgtttggaggatggcaagctgaagacatcgtcctatccatcatctgactgctccttgcaggccactccatccaaaccaaacgatccagctctcgcagatctcgtggagaagaagctgtccagctcgatagagcagatcgagaagatggtgcggcgaaacctgaagagaacttcgaaggccgctcagccttccaagaggaccatccagaggcgcgtcctgatgtccatgcgatgagctgagaaagctatctgctctgccatggcttcccttgtctgctgctttcttagcagcaagtcgtaaccaaccattacgccagttttgcctgaaggaggggtggggccgtgggggctcctggttctctgcttgaggaccccggggtatgcggcttactactagctctctgcttgagggtacacattatggtgactgcgtatttttaatttgagaaattagtagttctatgtatagttttatagtgtggaacggatatgagaaattagtagcaattgtgacttcgacagcttgaatactgttgttatggagtagttcctctatactgttgtttccgagtaacataacacagttttggccatcagtaatgga

SEQ ID NO: 3 - DNA, 684 bp, Zea mays
  source 1..684: mol_type = genomic DNA
atgcactcctccaggaacttcaagatgcttgaagtgaggactgcaagaaggccggctctcgccgacatctctggtggtgggttctttatgaggacagtagaatcgccaggagctgtgctggtgaatggtgctgtcaagcggccggctcggcagtttctatcaccttcaagcaacaaggagaatgtgccaccagtgggagctttcagggctacaccaaagaggaggacccccttgcctgactggtacccgaggacaccactccgtgacatcacatcaatcgtcaaggcaattgagaggaggagaagtcgtctgcagaatgctgcagctcagcagcagatccagtggacagaagacccttcccgatctgtggatccaataactccagtacaggcagagcagggtggtgtgccaacaactgtggatggtcaaggtgttggaagccctgcaacctgtttggaggatggcaagctgaagacatcgtcctatccatcatctgactgctccttgcaggccactccatccaaaccaaacgatccagctctcgcagatctcgtggagaagaagctgtccagctcgatagagcagatcgagaagatggtgcggcgaaacctgaagagaacttcgaaggccgctcagccttccaagaggaccatccagaggcgcgtcctgatgtccatgcgatga

SEQ ID NO: 4 - protein, 227 aa, Zea mays
MHSSRNFKMLEVRTARRPALADISGGGFFMRTVESPGAVLVNGAVKRPARQFLSPSSNKENVPPVGAFRATPKRRTPLPDWYPRTPLRDITSIVKAIERRRSRLQNAAAQQQIQWTEDPSRSVDPITPVQAEQGGVPTTVDGQGVGSPATCLEDGKLKTSSYPSSDCSLQATPSKPNDPALADLVEKKLSSSIEQIEKMVRRNLKRTSKAAQPSKRTIQRRVLMSMR

SEQ ID NO: 5 - DNA, 2817 bp, Zea mays
  source 1..2817: mol_type = genomic DNA
gaccgtagcagcgtagccagtagcccagtacccagtagtacccagtagccatctccatccaggctcaaaccctagggaatctacccagaaatcacaccccaagcagggtcctatcgtatcaagaagccgtcgcgggggccggatcgccgcggctcggagaggagcggagcgaaaatcgtgagcaacctttgctgttctccagtggatatgttcttgcatcttttcgggcggttcgtgattcgattcttctttgttggttctgttaccacagatctgcggacaggtaaggattcggaggcgggatcaggagctgagggaacctgttcgaggtgagaatcgaaggacagcagggggggtttggttggttctttcgtgggattttgttggttcagcctccgtctctttgttcttacgtgtttgactggttattttggttcgaatacctgccaaggatggggaatatgagtagagcggctgggatttgcatggttttgatggttttcttgaagctcttgtctgaactgttgctcttgtttgccttgtgtttttcttgcaggtggcctagactcgtagggatcgcgttgacattgggagaaatcacggagaggcggaaaccgagcaatagccaataggtgcgtagaatttggtgaataggttgctcacgtcctctctgaatatttcttgacaatctggcaatttcatccaaaaaaaatatttcacgcctccaggggaagggaagagcaacagtgcccggggcaccaagaattcgttgcatagatccaggagaagaacaatagaaatctgggcagaacgagagcagaactgtaagaaaatttatggtttcttggtccatttactgatgtgctgattcattgtctggttggttcttgaatcactgtctcaccgatatgagctacacaaacttctcgcaggactacaagatgcctcaattgagaactgccagcaggccggcgctcgccagcaactctgctggtggtttttttatcaggagaagggtggcatcaccaggaacttcccaggcaaagggcgccgccaagccgctggctcgacgggtccgaacacctgcagcgagggctaaaccaaagaggagaagccccctacctgactggtacccaagggtcccactccgtgacatcacatcaatcgtaaaggtggtgaactatttccctagactgtatttggatgttccataaatttctttaatattgtgtatgttctgtgctaatattatgccataaacttcaccatgctagaactaatattgtgtaaccatgatttgattgaggatgacagtagcatgaaattgcaggctaagcaaatcttttatgcttaatccagtgattaacataaaagttgttgtagttcaaatggattagcgatgattttgttaggccaatcagcacccttttgttttatttgattggattttgctgttctcagtttatgcagtcagcaatatgaatgtgcttaaaactagagaaaggtctgatgctcccctctttcaaaattaagttgctttagttttgacctaagccaaactcttctaactttgctcaagtttatagaaaaaatatttaacatatacaatttacggtacataatgaatgcgttatttaagacttatttcattgtggatttaatgaaattaagttgatgttgtgatgttggtgtgtttttctataggcataatcaattttaaaacggtttgacttagaacgataaaagacttacatttttgggaccgatgtagctagcagtatgcaataaggtcaatcttatcgatatattttgcaccatttaatatatattgctgctttgcaatcttgtaaatgcagtatcatcatgcactgaataacatatcatgttagacgttcactaacatttcagtatttcactataggtagtccatgcttttgtttgtcgatgcacactcaaaaattgcttgtccaccatgctactgtactaactgcaatttacaggctcttgagaaaagaaatcgcctagaggaggatgcggctcggcaacacatccaatcgaatgaagattcttcacagcctgtggatccaacaactgcagaacatagtaatcctgattctcaaagcacgcaaactcaagaaacaccgggtgctgttgcttctggtccaagctcaacttcagctgttgcaaaccgtgtgacttctgtggctgagggcaagcaagaggcaacggactgctccttgcaggtggctccatccaagccaaatgatccatctcccgctgatctggtgaagaaactgtcaggttcgatagagcagattgaaaagatggtgaggcgccatatgaaggaaactcatccaaaggctgctcagccttccaaggtagttgtccaaaggcgcatactgatgtccatgcgatgagctcagcaacgatttctcttgtctgctgcgttcttggccgctagtcaaggtatgtctagggatggggattcctggttctctgcttgaggacactgtggtaggagtaaaagagggatttctagcttctctgcttgagagcatggctatctgttggtacaacttgccatggtatgcttactgcatacgacttcagttggcttcaccagaaacgaaccactgttgttcacgtctttctgtttgtgaagtataagacttcactgcgtagtagtaaggcgtactagatatttgtggcgtttagttcaccgaaaagatagagcagttacgagttgtagtaacagcttgaattcttgacacttcaattgatacagttagccttgcttattgttccagattttggttg

SEQ ID NO: 6 - DNA, 1464 bp, Zea mays
  source 1..1464: mol_type = genomic DNA
gaccgtagcagcgtagccagtagcccagtacccagtagtacccagtagccatctccatccaggctcaaaccctagggaatctacccagaaatcacaccccaagcagggtcctatcgtatcaagaagccgtcgcgggggccggatcgccgcggctcggagaggagcggagcgaaaatcatctgcggacaggtaaggattcggaggcgggatcaggagctgagggaacctgttcgaggtggcctagactcgtagggatcgcgttgacattgggagaaatcacggagaggcggaaaccgagcaatagccaataggggaagggaagagcaacagtgcccggggcaccaagaattcgttgcatagatccaggagaagaacaatagaaatctgggcagaacgagagcagaactgactacaagatgcctcaattgagaactgccagcaggccggcgctcgccagcaactctgctggtggtttttttatcaggagaagggtggcatcaccaggaacttcccaggcaaagggcgccgccaagccgctggctcgacgggtccgaacacctgcagcgagggctaaaccaaagaggagaagccccctacctgactggtacccaagggtcccactccgtgacatcacatcaatcgtaaaggctcttgagaaaagaaatcgcctagaggaggatgcggctcggcaacacatccaatcgaatgaagattcttcacagcctgtggatccaacaactgcagaacatagtaatcctgattctcaaagcacgcaaactcaagaaacaccgggtgctgttgcttctggtccaagctcaacttcagctgttgcaaaccgtgtgacttctgtggctgagggcaagcaagaggcaacggactgctccttgcaggtggctccatccaagccaaatgatccatctcccgctgatctggtgaagaaactgtcaggttcgatagagcagattgaaaagatggtgaggcgccatatgaaggaaactcatccaaaggctgctcagccttccaaggtagttgtccaaaggcgcatactgatgtccatgcgatgagctcagcaacgatttctcttgtctgctgcgttcttggccgctagtcaaggtatgtctagggatggggattcctggttctctgcttgaggacactgtggtaggagtaaaagagggatttctagcttctctgcttgagagcatggctatctgttggtacaacttgccatggtatgcttactgcatacgacttcagttggcttcaccagaaacgaaccactgttgttcacgtctttctgtttgtgaagtataagacttcactgcgtagtagtaaggcgtactagatatttgtggcgtttagttcaccgaaaagatagagcagttacgagttgtagtaacagcttgaattcttgacacttcaattgatacagttagccttgcttattgttccagattttggttg

SEQ ID NO: 7 - DNA, 648 bp, Zea mays
  source 1..648: mol_type = genomic DNA
atgcctcaattgagaactgccagcaggccggcgctcgccagcaactctgctggtggtttttttatcaggagaagggtggcatcaccaggaacttcccaggcaaagggcgccgccaagccgctggctcgacgggtccgaacacctgcagcgagggctaaaccaaagaggagaagccccctacctgactggtacccaagggtcccactccgtgacatcacatcaatcgtaaaggctcttgagaaaagaaatcgcctagaggaggatgcggctcggcaacacatccaatcgaatgaagattcttcacagcctgtggatccaacaactgcagaacatagtaatcctgattctcaaagcacgcaaactcaagaaacaccgggtgctgttgcttctggtccaagctcaacttcagctgttgcaaaccgtgtgacttctgtggctgagggcaagcaagaggcaacggactgctccttgcaggtggctccatccaagccaaatgatccatctcccgctgatctggtgaagaaactgtcaggttcgatagagcagattgaaaagatggtgaggcgccatatgaaggaaactcatccaaaggctgctcagccttccaaggtagttgtccaaaggcgcatactgatgtccatgcgatga

SEQ ID NO: 8 - protein, 215 aa, Zea mays
MPQLRTASRPALASNSAGGFFIRRRVASPGTSQAKGAAKPLARRVRTPAARAKPKRRSPLPDWYPRVPLRDITSIVKALEKRNRLEEDAARQHIQSNEDSSQPVDPTTAEHSNPDSQSTQTQETPGAVASGPSSTSAVANRVTSVAEGKQEATDCSLQVAPSKPNDPSPADLVKKLSGSIEQIEKMVRRHMKETHPKAAQPSKVVVQRRILMSMR

SEQ ID NO: 9 - DNA, 16989 bp, Zea mays
  misc_difference 10801..10900: note = n is a, c, g, or t
  source 1..16989: mol_type = genomic DNA
cgagattacgagacaagagacaagtagacaacccagctctccagcggtccagcggtccagcctctcctagccgaggccaaaacccccataccaacacacacttcgcttctcgctgcctccgcggcggcgcgccgctccgctccgagccctcttcctcctcctcctctgcccgttcggtcgagaatgttctactcgcatcagcttctcgcgcggaaggcgccgctcggccagatatggcgcgtctgctcccttgatcgcacctcctatgcctctcgttgcgggctctaatcgcgctcgtttcccttccttgttccttgcaatgcgtttggttctatgccttatcctggcatcgacccacagtgatggccacgctcgcgccgatttggcggtcactaactgcgtttgcattgtggtttcaggatggcggcgacgctccactcgaagatcaaccgcaaacggctcgacaagctcgacatcatcaaaatctggtggggaaactggtcccagggattcgagttccgcacggttttgactgctatttttgtgttttttttatttgtttttgctgacttttgtttgtgtgtttcacttttagtgaggaaattttgaacccctcggtgcccatggctctgaggctctctggaatcctcatgggtgagttcgattttgcttgcgccacccaaaactgttcctcccattttttggatagtttttttttacgttccaatttccatggtcgaaaattcgaaatgcgtgagcaggtggcgtggtgatcgtgtacgagaggaaggtgaagcttctctacagtaagtttcttcctccctacatcctacttgatgttgattttgtttgaattcccagtgtttcatcatcccaattctaaaatcgatcatgtgttctctttctcgcagctgatgtctctcggcttctggtaagaactagttcaggatctgcccgagtgttttctgtgtcctggtcagtcgatttgaatatccttgcgaattcagttcgagttataccactaacttctcgtgcagactgagatcaacgaggcatggcggatcaaaccggtcacagaccccaccgtcctccccaagggtaaaactcaagccaagtaagtgtgctaccggtctcctccctttctaagcgtttatagatgttacatgctggggttgagggggttcggtttgagtaaggttgcactgcattcatgctgttgatgttgttattgatgagacttggtgtttgcttcttagctttcttaaaacagagattgttgtgtgtgcgtttaggctgtgatgtgttcatctggttgtttggattggcgaatgttgtgatctggctgagatgttcatggaagttcttaggagatagatatgtgttagtttgcttgaactgaattggtatctggtgcaatttgtgtgccgtgggaaacgaaaatgggattcacagctacatgttggcgattcccgtggtagtttgttgtgccttactcactgggcccttgtgcttgctgaagcactcacatccactgtctgatactctgatatctgatgtatgagtcccaatgatcttctgtcaactggcctaatcacttgctgttttatttttttatttatatttataattttgacttttgagatatcagaataggaggtggatggatttgcatttagcatattttggtgcaacagttactttctccttccaatatgtgagcatcttcgtgtcgttggagtatcttttggcatattctttagcgtgtccatttgctagctatacaatactgtacttgagaaataatgctatcttaaaagggttttcattacctttgagaactggaaatgaacatccacggccttgattaattcttttggcattaccattcctatggctgtagtataacccttggaaatttacttgagaaatgtaatcttttcttgatatagcacatttcgggttaaacttctatagataaatttgacctcattactactagctctttcatagccaaacacctaagtctctccccttgtgtaaatagcatggagtggatatgaaccggtgatatgttatgtggggcgccacttacaggcgcaggcctaaggataagagacctagttgagtttatctagagatattagtcaagaatatctaggggtaatagcagattagaagttgttgagattttttaggagaagtttaaggagatagagtcaagcagataggaggttgcggccatgcaactggcctatatatgtaatggaaagttatgaaataaaggaggaatgaattatctccaaactgtctttcttccctcctaatccggtctccctccttgctgcccaacatagatcagcacccaaccgatcgtccacgccacatgcgaagtcaggggccggcgcctgcgcccttgccacttcccccataccgtcggccacataacatcttggtttcaggagaccagccacgcacctacccaccaccatgtcctccatcccaagcgatccagtggctcaggccttggacgccatcaaccaaaggctggcaaccctggacaccctgcacaccactatacagcgcctagaacggcagcaacaggaaagctaggctgccattgatcgccttgattagcgccagcaggagatcatccctcacgggaaccaacgtccacgaggcgggcccaatgccaaccatgaagaccgcccaaccagattccatcatctggatttcccacaatatgacgagaagacggatccgctactcttcaccaacaaatgcgagtcattcttcttccagcagcgagtcgttctagaggacaggtatggatggtatcataccacctggaaaacgctgcacaacagtggtacatgcaattgcaccaggaagaagggacgccccccttggcaacgttttgccgagctgctgaaccttagcttcgggcccccgtccgagcctgccccctcggcgaactagcgagttgccggcgcacaggaacggtggacgcctacaccgagcacttcctagagctgctacccagggctgggccgttgagcatgaatcaaaagatccagctgttcaccctgggattacaggaaccactctccatcgacgtcgaactccaacacccggtcacactggaggtggccatgagcctcgccagagcctacaaacgccgggaacaggcagttgcagcagcacaggcgtcgaccaacaggccaccccgatctagtcgtggcttgcttccaacgctgtcgacaccccagctgccaccactcgctggctcagactcgtcatcaccattatgcgcccggccttccagcagcagcggacaggccatccgtcggctctcaccggaggagatggacgaacgccgacgtttgggactttgcttcaactatgatgagaaatttgccagaggccacaaccgcgtctgcaaacatctcttcctcaaactccacgaaggcgagagcgacgacggcaacacagaagaaccggccagcaacaaccccgtcatctccctccatgccatcgccggggtcacggccaacaagaccatgcaggttccggtcagtctgggcacagtcagcatcgtcgccctcatcgactctagatctacacaactccatctccaaagccaccgccacacgaacaggtctcccggtcgcgtaacgggggaacatgtgcatcatcgtggctaacggcgagaagctgccatgcctgggagtcttcagatcagggcccttcgtcatccacaacaccaccttctcggctgatctcatcgtcctgcccctggccgggttcgacatggtactcggtacctagtggctcgccacgctggggccgatcctctgggacttcgccaagttgtccatggcgttctggcgtgaaggacgacagatggagtggcgaggactcgcagagacaccacgaccgcgcctgctcgcagcaacaggccaggacgtcctcgacgctatgctcacctccttcgacgacctgttccgcgagcctcgtggcctaccgccgcagcgaccctgcgaccaccggatccatctactaccgaccacggcgcccatcgccgtccagccgtaccgatacccagcactgcaaaaggatgaattggagcgccagtgccacgacatgcagcagcatggattgatctgccatagcacctcgactttctgtctccggttctcttggtcaagaaatcggacgagacctggcgtttctgcgtcgactacagagccctcaacgaacgcaacatcaaggactccttccccattccagtcgtcgacgagctgcttgatgaacttcgcggcgccaagttcttcaccaagctcgacctccgctccggctatcaccaagtgcgcatggccaccgacgacagccacaagacggcgttccgaacacacgaaggcctgtacgagttcctcgtaatgccgtttggcctctccaacgcgtcggcgacattccagacgctcatgaacacggtccttcatccgttccttcggcgcttcgtcttggtcgacatcctcatctacagcagcacctggtccgaacatctgcatcacctgaggaccgtcttcaccgcgctcagcgaccactccctggtgctcaaaagatacaagtgctccttcggcgcagcgtcgacctcctaccttggccacctcatctcggcggaaggcgtggccatggacgttgccaaagttcaagcagtggttgattggctgccaccacgatcggtccgagcactacgcgagttcctgggacttgtgggctactaccgcaagttcataaagtcatatggtgaaatagcagcacccctcactgcgctactcaagaagaatggcttctcctggaccgatcagaccacaacaacattcctccacctgaagaaggcactcaacacagctcctgtgctcacccttccagacttaggacaagatttcaccatagagtgcgacgcctctggagccggatttggcgtcgtgctgcaccagggagcagggctggttgccttcttcagccgagcactggcacctcggcaccgcgacctcactgcctatgagcgggagctcattgggttggtacatgcggtacgacactgacgcccatacctttggggtcagccattctccattcggacagaccactacagtttgaagtacctgctagatcagcgattgtcacatcccccaacaccactgggtaagcaaactgcttgggtttgatttcagggtggaataccgatcagggcgcaacaacattgttgcagacgccctgtcgcgccgcgacatggaagacgctgcaatcttctccctgtcaggaccaacattccaggtgtttgatcaactccgccaggcagcagcatctcagtcggcccttgtcgcattgcgcgacgagatcctggtcgacacacgcaaaacaccttgggcgtttgtcgatggactcgtgttgttcaatggtcatgcatatctccctcccgagtctagcatcctccaagacatactcgtgtcgactcacgacgtggggcatgaaggcacagagaagacactgcaccggtttcgacgggattttcacactccaagggcaagggctatcattaaggacctggttcggcactgcatcacctgctagtgaaacaagactgaacatcttcatccagcaggcctccttgcccctctaccaataccaacgtcagtctggtcagatatatcaatggacttcatcgaaggccttcccaaggttgggggcaagtctgtcatcctgtcaatttattcagcagaaacaatagcatcaatcttcttctcggaggtagtcagactccatggcctcccatcaaccatagtgtcagatcgtgatccagtgttcacatccacattttggacaacactcttcaagttgatgggcaccaagctacacatgagttccgcattccacccccagtcggatggtcaaacagaaacaatcaacaagaccattggcatgtacctctggtgtttgacaggtgaccgtccccgacaatggctgcgatggctcccttgggcagaatatgtctataacacctccttccgcactgccctcaaggagactcccttccgcatcatatatggtagggatccaccagcattacgagagtatgacctgggagagtgccgagtaccagcagttacccagtccatgacagaaagagaagaattcttgagtgatgtacgcgcccgactggagcaggcacaggccgtagccaaacgtgcctacgaccgtggtcacagggcagtgagcttctctccaggtgattgggtatggttgcgagttcgtcatcgctctccagcaacgctctcagcggtgatgcgcggcaagctgaggccgcgctactttgggccatacaaggtggcggccatgatcaatgaggtggcttatcgcctcgaactgccatccacggctcggattcacaacgtgttccacattgggttactaaagaaatttctgggcactccagattctccaccaccactcccgccgattcatgacggtgctgcacagctgcaaccagcacaggctgaagattcgtcaagccagaggactttgctagatccttattcagtgggattcattgcctagctcagcagcaacttgggaagacctggacgacttcaggaggcaatatcctcattttcagctcgaggacgagctgatactcgatgggggaagagatgttatgtggggcgccacttacaggcgcagggctaaggataagaggcctagttgagtttatctagagatattagttgagaatatctaggggtaatagcagattagaagctgttgagattttttaggagaagtttaaggagatagagtcaagcagataggaggctgcggccaggcagctggcctatatatgtaattgaaagttatgaaataaaggaggaatgagttatctccaaactgtctttcttccctgctaatccggtctccctccttgctgcccaacatagatcggcacccagccgatcgtccacgccacctgcgaagtcaggggccggcgcctgcgcccttgccacttcccccctaccgccggccacataacatgatataggaaagattttcttttcgaagagtaagaaagtgatgtggcatgcttatctgacagccgaacttcatgcaggtatgaagcagtaacactgccagagatcaatatggtggtggaacagcccatgtttttctcagagcctgatggtgccaagttccgacgaatggtaatccttgtcacctgtgtatttcagaaaaaaaaactttatatgcatacagtatggtgcttatttagaataaccatcgcatgaacacctcacttttttctgactttggcctgattgtatccactcttctgactgaaatgtatcatgcatgcttagggattggaagatctggatgaacagtatgttcaagtcaatcttgatgatgatgacttctctcatgctgatgatcgtcatcaaggttggtctttctcatcttgcggattactctgtgtttgtataggggtgaatattttcaaactctacaatattttctcagctaaagcagtgaatataaccctggtcgataattttgagtctgggcttgcagaaactgatttattcaatcactttgagaggtaaagaaaagctgcatatttatttaaatttaccactcttttatcatcgctgctgcatgtttctttttcttattttgcttatagtacaaatataaatagtgccttgtatacagatttgacatagcagatgatgagaccacagtcaatatcactcctgatgagtacccacaagttccaagtacgctgattccgtcaccacctaggcaggaagacattcctcaacaagaagaaccgtactacgctgccccctcccctgttcatggagaacctcaacaaggtgaaaccaaacacatgatttactctctgccatatggaaacaaggccgtaacttaaaaaaatatagaatttccttttccccccaaaggacaatgatttttatgtcaattgtgcattgacatgtaatttcgcaagaggaacttgcatccaaaagttctccagatgatggttcttactggcagctaatggtcatttgcagactctattggagtattccaaaaacaattcacactttatctctcattgcctttgtacatactcaaatggctacagtaggacttttttaggattaaaggctagggagagcatctttcttttgacgagttggggaggcggggggtatagtttctgtcggaggggagaatccttcggccgagtggtggattgcacctgccctgatcctcagatgaggagtaggcttaggtgattgcctggccttctagatgaacacaagggtttagagtggttcaggtcgtcggagcgtattaccctacatccactgtgagttgtattgcttgagaactggggagtcgaaggtctgagtgtgaattggcctcccctgctacggaacaagtactctcccttttatagtctaaggcagacacattacaagggagttcaggccttgacaggtggacctaggagagtttacaatatggagcggtaaacaactggatagcgctgacaacgctagatcttcttgagctcgtatcccttcttgggtactactccagcttctggcgtccttatcttgccaccttgtcatgtctaatcggaacataggccttgatgtaggttgcgacgtagtctgatacgttgttgtgcgtagggtgtgatgaatggtgtcgtccagtcttctccgtccgtcccttcatgcctcggtaacacacgaggcacaatggaagtcataatgaaggacgcctaaacgacgcattaggtcaatgcccttgcctcggtaacatgcgggtaacagtaaaatccacacgttacaggcctgagatgtagcgtgcattaaatgccagggacacgtgttggggcgaaggctcaacgccccctcgctcgggaaaccacaataacgaagggtcaaggactggagggtgcgaaggcctgaagagcgaccacgcttcgaagaacacgaagagtccgaagactcgggcacccctttcaccaggacgggccagagcgaagaccatcgagaattccgtcggcggaggatatggactgcgaagaccatgcgctgtgatctaccgccaattgtaataggggggcccatgtgtagtcggcccacgctgtgagatctagctcgcaggccgctacgcaagtaggactgttagcggatcagcccgtgtttgtaattaattacgctgtaatgacccactgtaaccccccaagggggaatattccggggatatcgtaggtaaccgagggtacatttgtatttgacggggcagtgccgaccctcggttacctataaataccctcgtactgcaccattgtgggggacggagaaaaacaatattgccctaataatttgtgttaaacgacatcaaattgtttcctattcttccactgttcgagcttggctgaccgcgagttagcgagttccaacaacacgctacctgctattgatgcaggcaagtatacgtctcatctgtactcaatgctggcgggccctatgtcagaggctttcgccagacatcgcccgatggcttacactaggggcgtctggccacgtgttaacaccggacccttgcctaggtgaggggcagacctggggataagtggtgacactgcttcccatgtctttcctaaccatgggctcgagtcgtacctgaggtgattcgggctactgttgcccttgcctggacacgtggtgactccagtcctcacctggcctgaggtacgaatggtatctttcatacccggaatcgttctaaagacttggtgttgtaaccagagagcgccttctcctatgtctcctccagatccctttggttggggatccgttgggacacaaggagatctgtgtcctatttgcagggtcccaaacctatagctgaggttgttgattcctccctttatggggcttgatgcagacgacgacccagccctggaggcaagcctggcctggtcatgactccatacggtgggccttagccccgtgctgggccgactgcccgtgaaatccttaacagtggcccccaatgactcaccttgacatagcaggagtgggtaccccgttttcagggtagcgacagtggcccctgggctcacctcgggtgaggcggcaaacccgcaggtggggccataccgtcttgttccagaaatgtcaaccttgtgcagcagcttcgtgcatgtagccttcacgttgagtgggttgcttgccggcacactgttattgctgcatgaagacaaagaaagtggtactgccctcgtgtttatccagttatgggaaggcaaggattattttcttttccgccccagccgctaaggctccctgaagtttttcccagttgtgggggattgcttttagtccagggagctactccggagttcgacttaggaatagtgcttaggagcatatggtattccgtgaatggtttaggagtagtgcttagcggtgtaccctcgagatcctagctttgttgtactcattctattgaattatgtagccgggttgttgctaggcagagtttataagatccaaccttaattttgaatgaggcgtaggcccgttaggtacctagtccaaaaagtactaagccagctattctgggacggtggaaggtgtaggcgtacgacacaggaccaggctaagcggctacctagctccggaccacctgctagaatgtggacttttcctttagaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctatcgtcttcttggagcacacaccggtctctactccagccgcaacattcgcaccatcatgacgttgcgactgcggggggatttcaacccgtcggcttctaccttcggcctctactccagtctcatcgtgtgtggtgcccccgttgcgactgcggggggatgttagactatgtgtgtgtgagtgtgtgtgggtcggcaccactgttgggctgccggcccattagggttagggtttcctgtgtctctatatattgtaacctcatctattatcaatacaacagttcatcttccctcatttccaactttgttttttttgttggttgactagttacctgatatattacaaactggctgctcgacataatttaagtacatgtgttgttgcatttatgccatatttgaagcatcttgctgcaattactgaaagttctcttgcagggggaccagaggaccaagaggaacagaagatgaaggtaaacatgatttatcaagccttattacatctagaaaatagtatgaaaagggattacttgtttacttgagctagcacttttacacacagcagcaaccaccgaaagcatcaaagcggaaagcacgttgggaagtcccacgagtaattatggacaataaccagatgatgatcccaggaaacatatatcagacatggctgaaggatgcatcaagccttgtctcaaaaaggcgtaaacttaacagcgtatgtttggctcgactaacctattttcctggagatttctgtaccgatgcattctaataaacaacatttatctgatttatgcttgcagaattttaattttattcgatcaaccaagataagtgatctcatgcacatccccccggttgctctaatatctcatgacaacttgttctcagaattatgttatcctaagccacttatgcagctctggaaggactgcactgaggtcaagtccacaaaagcttcttcaggtcacttcataatttgtaatatcatgtcactcttgactaaagaaaccatattattttaacatgctaaaggccaattgaaaaaccttaactgtttcttactttcttaaaggagggcagcgatcatcatcacaagaaccacaaccgaaaaactcaccacctcaggttattctagattcttttcagaaaagaaaaacacccttccctagtgactcatcataattatcattaaatttgtccctggtgagtggttgaattttcaggctgggggagagtatgagatggaaacaggtggtctgccgatggatttaacagatggcattgaaaaactcagggcaaacatgagtgcaaaatatgacagagcttacaatattcttcacagtgaccatagtgttactcctggaagtcctggtaagttgaaaagtgaataagtttaatttctgtctgtgcttatatagacagttgatgacgtaaatctctagcagggttaagtcgcaggtctgcttcaagctctggtggctctggatcggcgtttatacaattggatccagaagtacaattgccatcaggaagtggaaggtgactgttgacttatagtaacagcagcatgtcacctatatactgaactatgactatgtgatattttggtactgaaagaacttgtccatctaaagttgttttcgtcatcacaacagaacctcttattgtatatgctcgtaactagtgagtgcagaaactaaagtgtgtaatctccttgatgaagtggcaggcaaataaaaaaaatgtctagctccccactgtccttcagtgtctttgttagtttcttaaagtgaccatgtcatggttattgaacacaggtccaaaagggggcagcattcatctgcaagaagcttggggaatcttgatactgttgaggaagattttccactggagcaggaagtgagggacttcaagatgagaaggctttcagattatgtgccaactcctggttggttgcattctgtcctttgctatatataaacatgtgaaactgtctctggacgactggaacataaactgtttcaaccaacatagaagttaacaattttactccaactcctggttggttccatttctgtcatttatttactctgttatatataaacatgtgaaactgttttctggagcatttgaaactgtttcaaccaacatagaatttaattgttggcattcgtttcagacctactggaagaaactgaaccgactcaaaccccatatgagaggcgctccaatccaatggacaagatcacagaaacaatccagtcgtaaatatccttaatttgtttgctcatagtgcctgggtcataaattatctgatgctagagttatcttcatgcaggcatctcaaacttcatttcgataccccaggtgtcccacaatctgaatccttaagtcatttagctcatggaatgaccaaagccagggcagcccgactattctatcaaatagctggtatgtattccctccgtcccaaattataggtcattttttgtttttctaggtacataacttttgttgacctataatttgaaagctaggtagtatttctctatggtgctttgactggaaaatgaagagtactgttgttaaagtaaataaaaaactagtagtggtcactgagatgatgttaaacagttatgatagctttactggcttagtgatggtttctgtaattaggttgggttacaattaaacaccattcaagcctgctttccttttactgctaggaaatttggcacaatttcctcaaatttgttattttagtgaccgtgtaacgttttctcagttactcagtatggtgcaaataactatttttttatttttttataagatggttgttgctctacctttgcattgtagaagtttaaaaaacgcgaaaaaaactctacctgagttagttacttggtctgagtagcactccttaagttggttatatggtctgagcgaacactcctcaggttttaagttcgactcacatggggtatggtgtcgttgtgtacgggtgtgacaggggtttgaaggttttttcgacctgtgcaaggtcttcttattaatataatgtctggaggttgtgtttccctccgcaggtcaagtttttatgtgaagtttaagaaaaaaacagtttggaaactcaaatcccctaggaatctctacatttttcccaaggtgaacaatctcatggataccattggttttcttttcccgggttcgtctccccttagctccaaactagcccttatcgaggagggtgacgcattagttttcaggtcttggttgtgtccaggtttgttgttgatgtttagctggatgaagttgttagttattagagccattgtcaaaacattgccagcaacgcataactggtttctctcaattgtggttaaaaagagaagtttcacacaacccaatactaggccgcataagggtccgttcgttttctctccaatccggggattgtggatgagtttcaatccatagcaagtcaaaatctctactatttttttttaatcttattcaatatatgtgatatagaaataaccgaacaaggcctaactgggaacacggagtttgcttaaaatatttgacataagaggaataacggtgaatctgaagtttatggtatttcattgtaattgctagaatggagcttattatttaatgggaatgcttgttctgcagttttggcaacttgtgattacatcaaggttactcagctggaacggaaaggagatgaactgtacggagacatcttgatctccagaggattaaagatgtgacttggtgatgttggaaacaattttgctgcggtggttcccggtgcacatctcgtgtttatattacagaatgcttacatattgcataggatgtcttgtagttaacacataatctatacatggtgatctggtgaaagaaagggagcgagatatgaggcagtagttttgtgttatgtggcaagcaaaattctaagctgttattttagaggtttcataatttaagtacgaacatgtgttatactattatgaaactaagttgtcctgaaagcacttactgaaagggagaaatttacatggtaccttgtcaacgatgaaacatacatcggaggacaacataaagatgcacactgtatcctgctcttcggctgcttcatgttacgatacctgtccatttgccctatctttcattttttatgggtggctttgtcctattctaaatgcaccttacttagcaatctgatattttttcgcctggaaataaatcacatgtatgactgatgtatctctgtctctaatctctaatatattaagccaccagtttcaacggttgtgacttctttttacaaagaatcccttgtagttcgtcaaaataaacctgcggtcctagatcatttcacatccctatcctatttgttcgtagagtagcgttctcgcaactgtcactctagcaacaaaccccgtccatttcgcaacaacctctgcccgcttcttccgtcgttgcaccgccatcggtgaggaggattagggcgcccgccttcggatatggatgctcactaattgcctgtttggttgggttctccccgtagcccggctgcatgagacaggccggcgggagcttggctccagagatgcgaccaatttgctcgcgcctgcagagcctagtccagggtgctttaagtattgtgcgagtctggctccacatctgcacgcaggtaaccaaacacatacttcgcatgcgtagagcctgattgacaacaaccaaacaccagctcattatatcccgcgatgcaagcaccagcaaatacaggcaaccaaacatatggtaagtgatataggtggtgtcgagtccagccaactctgaacctcctcgttgcccctagggaccgggagtccgcctcctagctcactcgtcttctactctggcgcgagcttttacaaagaaccccttatatttcttcaaaatcaatccacggtcccagatcatattccatctctatcctatttgttcgcacagtagcgttttcgcatatgtcgctctagcaacaaacctcgtccatttcgcaacaaacttcgcccgctttttccgtcgttgcaccaccgctggtgagaaggattagggcgctcgcctccggataaggatgctcactggatgataaaagttgtgtcgaatccagccaactctaaacctcttcgtcactcctagggaccaggagcctgtctcctagctcacccgtcttctgctccgctcctcccaccccatggcgacgtggcatcaacggtagatctacctagcgtgtgcttcatctggtcacagttgttctcgtcggatctattattggtagagattgctctaattgtcgtgtttttcgcaggggacgtccactcaccaggatacaagcttctcaaataagcaagccaagctgctcaagacgtagaagttcgcgtccgagtttgaccatctggtcagttcattctttcactcataccctttgtttgtcgatacttttagagatatatagctattgaatgcagaggcttcctctgattcggcgattttgtgcttggtaggtggatacgtcaaaggtcaagttggacgtgatgaagctctggattgccaagcgggtcattgagctacttgggttcgacgtatgtttctgatccatgcagaggtagttgcatttttcactgaaaattagcccaggtgctacggtcatcttgttgtttgtgaccatgttccctttttcttcttttttgttgggtctttacttaacgagaaccctaactcatttaccattgtgataggaccaaaattgaagtccaaactttgcactacatctacagaacgtttgctttggtgtcccgttccaatatgttgcatgcttagagtttaagacacgatgctcaagcaatgtactggcacacagagttgcaattgcaggcagctgcgcttgcactacacatgtgcaaacaattccaacagaataatatatagtagtaccccctatgcagcacccgctgtaatacatggatcacactatttcatcagttataaataccatgagatgaatccagaagaaaaaaaagttgtatgtt

SEQ ID NO: 10 - DNA, 2514 bp, Zea mays
  source 1..2514: mol_type = genomic DNA
cgagattacgagacaagagacaagtagacaacccagctctccagcggtccagcggtccagcctctcctagccgaggccaaaacccccataccaacacacacttcgcttctcgctgcctccgcggcggcgcgccgctccgctccgagccctcttcctcctcctcctctgcccgttcggtcgagaatgttctactcgcatcagcttctcgcgcggaaggcgccgctcggccagatatggatggcggcgacgctccactcgaagatcaaccgcaaacggctcgacaagctcgacatcatcaaaatctgtgaggaaattttgaacccctcggtgcccatggctctgaggctctctggaatcctcatgggtggcgtggtgatcgtgtacgagaggaaggtgaagcttctctacactgatgtctctcggcttctgactgagatcaacgaggcatggcggatcaaaccggtcacagaccccaccgtcctccccaagggtaaaactcaagccaagtatgaagcagtaacactgccagagatcaatatggtggtggaacagcccatgtttttctcagagcctgatggtgccaagttccgacgaatgggattggaagatctggatgaacagtatgttcaagtcaatcttgatgatgatgacttctctcatgctgatgatcgtcatcaagctaaagcagtgaatataaccctggtcgataattttgagtctgggcttgcagaaactgatttattcaatcactttgagagatttgacatagcagatgatgagaccacagtcaatatcactcctgatgagtacccacaagttccaagtacgctgattccgtcaccacctaggcaggaagacattcctcaacaagaagaaccgtactacgctgccccctcccctgttcatggagaacctcaacaagggggaccagaggaccaagaggaacagaagatgaagcaaccaccgaaagcatcaaagcggaaagcacgttgggaagtcccacgagtaattatggacaataaccagatgatgatcccaggaaacatatatcagacatggctgaaggatgcatcaagccttgtctcaaaaaggcgtaaacttaacagcaattttaattttattcgatcaaccaagataagtgatctcatgcacatccccccggttgctctaatatctcatgacaacttgttctcagaattatgttatcctaagccacttatgcagctctggaaggactgcactgaggtcaagtccacaaaagcttcttcaggagggcagcgatcatcatcacaagaaccacaaccgaaaaactcaccacctcaggctgggggagagtatgagatggaaacaggtggtctgccgatggatttaacagatggcattgaaaaactcagggcaaacatgagtgcaaaatatgacagagcttacaatattcttcacagtgaccatagtgttactcctggaagtcctgggttaagtcgcaggtctgcttcaagctctggtggctctggatcggcgtttatacaattggatccagaagtacaattgccatcaggaagtggaaggtccaaaagggggcagcattcatctgcaagaagcttggggaatcttgatactgttgaggaagattttccactggagcaggaagtgagggacttcaagatgagaaggctttcagattatgtgccaactcctgacctactggaagaaactgaaccgactcaaaccccatatgagaggcgctccaatccaatggacaagatcacagaaacaatccagtcgcatctcaaacttcatttcgataccccaggtgtcccacaatctgaatccttaagtcatttagctcatggaatgaccaaagccagggcagcccgactattctatcaaatagctgttttggcaacttgtgattacatcaaggttactcagctggaacggaaaggagatgaactgtacggagacatcttgatctccagaggattaaagatgtgacttggtgatgttggaaacaattttgctgcggtggttcccggggacgtccactcaccaggatacaagcttctcaaataagcaagccaagctgctcaagacgtagaagttcgcgtccgagtttgaccatctggtggatacgtcaaaggtcaagttggacgtgatgaagctctggattgccaagcgggtcattgagctacttgggttcgacgtatgtttctgatccatgcagaggaccaaaattgaagtccaaactttgcactacatctacagaacgtttgctttggtgtcccgttccaatatgttgcatgcttagagtttaagacacgatgctcaagcaatgtactggcacacagagttgcaattgcaggcagctgcgcttgcactacacatgtgcaaacaattccaacagaataatatatagtagtaccccctatgcagcacccgctgtaatacatggatcacactatttcatcagttataaataccatgagatgaatccagaagaaaaaaaagttgtatgtt

SEQ ID NO: 11 - DNA, 1809 bp, Zea mays
  source 1..1809: mol_type = genomic DNA
atgttctactcgcatcagcttctcgcgcggaaggcgccgctcggccagatatggatggcggcgacgctccactcgaagatcaaccgcaaacggctcgacaagctcgacatcatcaaaatctgtgaggaaattttgaacccctcggtgcccatggctctgaggctctctggaatcctcatgggtggcgtggtgatcgtgtacgagaggaaggtgaagcttctctacactgatgtctctcggcttctgactgagatcaacgaggcatggcggatcaaaccggtcacagaccccaccgtcctccccaagggtaaaactcaagccaagtatgaagcagtaacactgccagagatcaatatggtggtggaacagcccatgtttttctcagagcctgatggtgccaagttccgacgaatgggattggaagatctggatgaacagtatgttcaagtcaatcttgatgatgatgacttctctcatgctgatgatcgtcatcaagctaaagcagtgaatataaccctggtcgataattttgagtctgggcttgcagaaactgatttattcaatcactttgagagatttgacatagcagatgatgagaccacagtcaatatcactcctgatgagtacccacaagttccaagtacgctgattccgtcaccacctaggcaggaagacattcctcaacaagaagaaccgtactacgctgccccctcccctgttcatggagaacctcaacaagggggaccagaggaccaagaggaacagaagatgaagcaaccaccgaaagcatcaaagcggaaagcacgttgggaagtcccacgagtaattatggacaataaccagatgatgatcccaggaaacatatatcagacatggctgaaggatgcatcaagccttgtctcaaaaaggcgtaaacttaacagcaattttaattttattcgatcaaccaagataagtgatctcatgcacatccccccggttgctctaatatctcatgacaacttgttctcagaattatgttatcctaagccacttatgcagctctggaaggactgcactgaggtcaagtccacaaaagcttcttcaggagggcagcgatcatcatcacaagaaccacaaccgaaaaactcaccacctcaggctgggggagagtatgagatggaaacaggtggtctgccgatggatttaacagatggcattgaaaaactcagggcaaacatgagtgcaaaatatgacagagcttacaatattcttcacagtgaccatagtgttactcctggaagtcctgggttaagtcgcaggtctgcttcaagctctggtggctctggatcggcgtttatacaattggatccagaagtacaattgccatcaggaagtggaaggtccaaaagggggcagcattcatctgcaagaagcttggggaatcttgatactgttgaggaagattttccactggagcaggaagtgagggacttcaagatgagaaggctttcagattatgtgccaactcctgacctactggaagaaactgaaccgactcaaaccccatatgagaggcgctccaatccaatggacaagatcacagaaacaatccagtcgcatctcaaacttcatttcgataccccaggtgtcccacaatctgaatccttaagtcatttagctcatggaatgaccaaagccagggcagcccgactattctatcaaatagctgttttggcaacttgtgattacatcaaggttactcagctggaacggaaaggagatgaactgtacggagacatcttgatctccagaggattaaagatgtga

SEQ ID NO: 12 - protein, 602 aa, Zea mays
MFYSHQLLARKAPLGQIWMAATLHSKINRKRLDKLDIIKICEEILNPSVPMALRLSGILMGGVVIVYERKVKLLYTDVSRLLTEINEAWRIKPVTDPTVLPKGKTQAKYEAVTLPEINMVVEQPMFFSEPDGAKFRRMGLEDLDEQYVQVNLDDDDFSHADDRHQAKAVNITLVDNFESGLAETDLFNHFERFDIADDETTVNITPDEYPQVPSTLIPSPPRQEDIPQQEEPYYAAPSPVHGEPQQGGPEDQEEQKMKQPPKASKRKARWEVPRVIMDNNQMMIPGNIYQTWLKDASSLVSKRRKLNSNFNFIRSTKISDLMHIPPVALISHDNLFSELCYPKPLMQLWKDCTEVKSTKASSGGQRSSSQEPQPKNSPPQAGGEYEMETGGLPMDLTDGIEKLRANMSAKYDRAYNILHSDHSVTPGSPGLSRRSASSSGGSGSAFIQLDPEVQLPSGSGRSKRGQHSSARSLGNLDTVEEDFPLEQEVRDFKMRRLSDYVPTPDLLEETEPTQTPYERRSNPMDKITETIQSHLKLHFDTPGVPQSESLSHLAHGMTKARAARLFYQIAVLATCDYIKVTQLERKGDELYGDILISRGLKM

SEQ ID NO: 13 - DNA, 5860 bp, Zea mays
  source 1..5860: mol_type = genomic DNA
cactgatgctcctggaactgcagtgacggttcggccgtgtcaaattctcgggtgtagggtgtacctctggccaagtgactgatattctgaaaaacatacggacttctagtataggagaagaggaatcctaatcctagtaattatatcttagtagatatgtgcttagtctcttgttaattttttgtcttgagaggactatatatactgataaagtgataatttattcagagaataaagaatgggctccatgctttgtctatgtattctcttcttgagggcaatttacgcctcttgagaagggtggcttcgttgttgggctcttgaaggcccacgcagtccaacggagcccactaagcctaattctgtttcttcgcgatcgattagctgattagctccatggcggcttcgaaaaaatcaaaataaaaaactagcgccatggcggggagggataagaggcgacgagcggcgccgctcgaaggcgacgagcagcagttgcggcggaggctggaggaagcggcgctcctcctccgcaggatcaaaggttttgttactcatctcctcgatcgcctgcccttaaatcttccctctctcctgtagcgtctggagctgacgcgcggttggggatggtgcagggcttgtgcgctggatcgtcgaagaggttgccgccggccgctccccgtctatagtgctccaccggtaccggaactattgctcctccgccgactctgcgtccccgtccccatggtttagcttccgcgcccgctcccgggatcatctccttcccgaccaattcaccgtcacatccctcgaacaattttcacccaaatgtgtgacatccgtagaccctgggcgcgcgctcgtgcgtgattgcagtgcctgcagctacgacatccccgtcggcacggacgtcctctctctgctccacaaggactaccacacctcccgcctcagtacgaatacatctaaatctttgcatttatgtgatcacactaattactttctgatgcgttgttcatgcgtagatgtgctcctgagggtgctgttcgtggtgcagcagctcctgcagcagaacaagcactgctccaagagggacatctactacatgtacccctccatcttcgtaggtctgtgctcagtaggtgcaatgtttgctcctcattccattatacgggattactagaatttgcacatatcttggctccctgcagaagtagcagttgttgaccgtgccatcaacgatatctgcatactcttcaagtgcagccggcacaatctcaacgtggtaggtccataggtgccactagtctgctgtcttccctgaagcttccattgtgtattgtagaaaggacttgtgccatcaacgatatttgctccttcactaatatgcagcaccatgactattacatactgtcttaatttcataaccttttatctgactgaatactgtgctcccttggctctctgcaggttcctgtagtgaaagggtatgaactgtcatagagttgaagctctgaagatatgtccgtttgttattcttctattattttacaacgcttcatttttttataacttaaataaacccgtttttttttgttccttaatcatcctgtgtgcttctcaccattatataaagaaagatgatttggatgtgtttgatgagctagtggggttgggaacctgggataccgttctttcaatgtagtcttagccgtataggtttatgcatatgaagaggaggaagaaatgatgccatgatggatctgttgttcttcccagtgtcagttcacaactttgagatatattttttttgatacagaacatgtctgaattcatgaagagttctctttggtttatctgaataaaaaatcaatatagcataagtgttaactaaattcactataggaatttggaaattctatatgatttccgtattacacttgctgttacttgcattgtttttgctaggtttgcgatgttatattcatagaactatttgttatttattcagttgtttaattgtgtgcacatgtctattcccttcattatcctagtttggtgatgggctggataagatttatggagggcgaaaagaaagtgtattgtataacaagcgtcaatgctgtaagcttttatgttctctgccccctaattttgctaactggtctattgataagatttcatttaatttgtttgctttaccttctgtatattcaggctttctccattcctgttgacattgaggcaatcaaaggtttttccatcaactttaggaacacaacccctttgtgcttttgtgaaatgtgtagaagggagggggtagtgaaaaatcaactctttccttgatgttttgaagcacactgggcaaaaagtcactgatcattttttgctcttaaatggtaccatagatcttcaattttttgtttctttttttaaaagaaattcataatcttacattctgaattaaataactactccccctcttccaagttatagtttactacttcactctagctttgtcccaaatcaaacctctctaacttcactttagttatatgttttgataagagaagatatccggtgcacatgaaagattggtgcacatggtgcacatattaaatcatcaccactcaatttagatctaacgtctctagttgtttggtatattttgctaaggacaccctccaacgtggtggtgtgtagtgatggaaggtattatttgtaaattgaatgatcaactaaagacgttagatataaaataagtggtggtgatttaatatgtgcaccatatgcaccagatatgtcctcttttgataatgcatttattgaacttctaggtgataatatattgttctaaaacttgatcaaagttagagaagtttgacttaggacaatgctaaagtgaagaattatttgaaacatcgggagtaccttttatcttttccagtcaaattttgtaattgagatattatatggttgtttaacaactcggatttagacgtgcctacaccagtacacctctatagcaaagcttttgatttcctcccttgataccctgaattgcattatagtgagctgaagttgtcttattctttcttctcttgcagatgttgttagtgttgcccactacatacttgtggttgagaaggagacaggtctttttttttttgcatttaataatttccgctctttgtacaatatgcttcagtagtccctttgcagtgttccagcgtttggccaatgacaagttctgtgaaagaaatcgctgcattgttattacagtaagtaacaactctcacccgatgttatcaattttcataattttttctctagtttcatgatattgcattgtcaacccagggaagaggctatccagatattccaacaagaaggcatgtgctctttattcattaagttcctcttgcatatttctcaaccgctatcccaattttacattctttttccatcctagattcctgcggtaccttgttgaactgctgcatctgcctgcttattgcttagtggactcagatccttatggtttcgacattctggctacctataaatttggttccttggtaggcatgatttatctgatgctgctatgctcatctatatctattgtctatgaacttaacttccttagcagattcacaggttaaaattgcaggtaacgaatccatggacatgatcatatactgcacctttcgtcctaaatcaacagcttgtctggcctctttgcatttgaaagttctattactttatgaagtcccgttagcaataaggctgtgtattagagcagttcatttttcccctcaaaataaacatgattatgctcgttttgctcatcttatcacaagcatgaccaaaatatttctttttgctctttgaaacccaagattatgacatatttacttgtgttaacttaccaacctctgctacttgcgtaatttcattcttcaagcaattggcacatgatgcaaacttgttgcgtgtccctgatatacggtggcttggagttttcacatctgattttgaagaatattgccttccagactgctgcctacttcgtttgtcacctgaaggtgaataaactgtagtgattcaagtggcatacatacttttttttggaagatgttccttagtttcttgttgtatgtcaaatgccaacctgtgaaatcatgtgttctgctgcgcagataggaggaaagctgaaggcattcttgctaggtgctatttacacagggaagccccagaatggaggtaatgaagtaatgaaccagctttggagttttactaggtgtattacaattatatcctccatgatatcattgtgatctccaggtcaagatcgaggtctattagtgcatggagttttactagagctttcgtggaatctgatttgtacagtcatcattcttatgatttagttgctggagtttgagtttcttcagcaccagatctttagcttcaatgactgtatagcttcctgtttttaccagtaaaggcacaatagtttgaagcactttatattttaagatattaaacgcatcatttcctatcaagaattctgagttattttttacattttctccctgaatttcacatgtattctctaatttgacaggtcagagttggaagcaatgctgcaaaagggtgtcaagtttgagattgaggcactatccgcaaattccatttcctttttatcgcatgagtacattccccagaaaatcaaacaaggcatgcatttataagattggacattcttgtatccgtccaggatgtgtcatagagtacaatctttttttgtctgattatagttatatccggagacatatcatgtgcaaacactaaggatctgtttcagaacataacataatatttccttaatcctaagataatatcgtggtatttgagcataacatgttattttagactaaaagatatttgggagcatatctaaaaactatgtattttaaaccatggttttactaatactttttgtttgtacataactacgtcttctcttctacactataactgaaatactgtgtttctaaacgaatgttggattagactgatataaaatataccatatgtatgtcatgatatacaataaactgtagtattataaactgtggttttaagaaacagagtttccaaataggccccaaggatgtgtttggtttgattatagttatatctcgagacatatcatggatgtgtttggtttgaggacattcctcactttttttatttgatttgtggattagaatgagggcatgtttggttctatgaggctaaactttagtctctcttttttagtccttaaattacaaaaaaaaaggaccaaagaagagaggaaatctgatgtattaccttttagtccccaatatttagtaatttgaggagtaaaatgaacaaccatcaagttgatctatcaccatctcatcctcacaagcaacgtgccaccgtgaaactcatctgattattatatatttatatgtcacagctgcttccaactactgctctcgcccattcctggagcaggcggcttgaacggatgtaaagtttttgttggtacttaattgatagtgcagtacgatggttcaggatggcgtaaaactaaaacaggtaatggtagtagtaagtagcgttatgatccccttagttattttgtttcagcctgcagttattttatatatgacctagttagtttaaaactgaactgattaggtgcatcatatataaaagaaaagagaagcatatatatatatatatatggctcacttttgtggtatcagaaagcaacactgtttgtcagcataaaagataacaaaagtagcgttattttcccgccaga

SEQ ID NO: 14 - DNA, 1307 bp, Zea mays
  source 1..1307: mol_type = genomic DNA
tcttcgcgatcgattagctgattagctccatggcggcttcgaaaaaatcaaaataaaaaactagcgccatggcggggagggataagaggcgacgagcggcgccgctcgaaggcgacgagcagcagttgcggcggaggctggaggaagcggcgctcctcctccgcaggatcaaagggcttgtgcgctggatcgtcgaagaggttgccgccggccgctccccgtctatagtgctccaccggtaccggaactattgctcctccgccgactctgcgtccccgtccccatgtgcctgcagctacgacatccccgtcggcacggacgtcctctctctgctccacaaggactaccacacctcccgcctcagtacgaatacatctaaatctttgcatttatgtgatcacactaattactttctgatgcgttgttcatgcgtagatgtgctcctgagggtgctgttcgtggtgcagcagctcctgcagcagaacaagcactgctccaagagggacatctactacatgtacccctccatcttcgtagaagtagcagttgttgaccgtgccatcaacgatatctgcatactcttcaagtgcagccggcacaatctcaacgtggttcctgtagtgaaaggtttggtgatgggctggataagatttatggagggcgaaaagaaagtgtattgtataacaagcgtcaatgctgctttctccattcctgttgacattgaggcaatcaaagatgttgttagtgttgcccactacatacttgtggttgagaaggagacaggtctttttttttttgtgttccagcgtttggccaatgacaagttctgtgaaagaaatcgctgcattgttattacaggaagaggctatccagatattccaacaagaagattcctgcggtaccttgttgaactgctgcatctgcctgcttattgcttagtggactcagatccttatggtttcgacattctggctacctataaatttggttccttgcaattggcacatgatgcaaacttgttgcgtgtccctgatatacggtggcttggagttttcacatctgattttgaagaatattgccttccagactgctgcctacttcgtttgtcacctgaagataggaggaaagctgaaggcattcttgctaggtgctatttacacagggaagccccagaatggaggtcagagttggaagcaatgctgcaaaagggtgtcaagtttgagattgaggcactatccgcaaattccatttcctttttatcgcatgagtacattccccagaaaatcaaacaaggcatgcatttataa

SEQ ID NO: 15 - DNA, 1239 bp, Zea mays
  source 1..1239: mol_type = genomic DNA
atggcggggagggataagaggcgacgagcggcgccgctcgaaggcgacgagcagcagttgcggcggaggctggaggaagcggcgctcctcctccgcaggatcaaagggcttgtgcgctggatcgtcgaagaggttgccgccggccgctccccgtctatagtgctccaccggtaccggaactattgctcctccgccgactctgcgtccccgtccccatgtgcctgcagctacgacatccccgtcggcacggacgtcctctctctgctccacaaggactaccacacctcccgcctcagtacgaatacatctaaatctttgcatttatgtgatcacactaattactttctgatgcgttgttcatgcgtagatgtgctcctgagggtgctgttcgtggtgcagcagctcctgcagcagaacaagcactgctccaagagggacatctactacatgtacccctccatcttcgtagaagtagcagttgttgaccgtgccatcaacgatatctgcatactcttcaagtgcagccggcacaatctcaacgtggttcctgtagtgaaaggtttggtgatgggctggataagatttatggagggcgaaaagaaagtgtattgtataacaagcgtcaatgctgctttctccattcctgttgacattgaggcaatcaaagatgttgttagtgttgcccactacatacttgtggttgagaaggagacaggtctttttttttttgtgttccagcgtttggccaatgacaagttctgtgaaagaaatcgctgcattgttattacaggaagaggctatccagatattccaacaagaagattcctgcggtaccttgttgaactgctgcatctgcctgcttattgcttagtggactcagatccttatggtttcgacattctggctacctataaatttggttccttgcaattggcacatgatgcaaacttgttgcgtgtccctgatatacggtggcttggagttttcacatctgattttgaagaatattgccttccagactgctgcctacttcgtttgtcacctgaagataggaggaaagctgaaggcattcttgctaggtgctatttacacagggaagccccagaatggaggtcagagttggaagcaatgctgcaaaagggtgtcaagtttgagattgaggcactatccgcaaattccatttcctttttatcgcatgagtacattccccagaaaatcaaacaaggcatgcatttataa

SEQ ID NO: 16 - protein, 412 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLSTNTSKSLHLCDHTNYFLMRCSCVDVLLRVLFVVQQLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETGLFFFVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSHEYIPQKIKQGMHL

SEQ ID NO: 17 - DNA, 21 bp, Zea mays
  source 1..21: mol_type = other DNA
ttctcctcctctcaattgcct

SEQ ID NO: 18 - DNA, 21 bp, Zea mays
  source 1..21: mol_type = other DNA
tcaccatcttttcaatctgct

SEQ ID NO: 19 - DNA, 600 bp, Zea mays
  source 1..600: mol_type = genomic DNA
tccatccagctccaggcgttccagaggccctgatacaggagatgttccagcacaacccaggggtgtccagggagggtctcggcctgtacataagccagaagctggtgaaaacgatgagcggcacgttgcagtacctacgagaagccgacacctcttcgttcatcatcctgatagagttcccggtcgcccagctcagcagcaagcggtccaagccttcgccaagtaaattctgacactgatgctcctggaactgcagtgacggttcggccgtgtcaaattctcgggtgtagggtgtacctctggccaagtgactgatattctgaaaaacatacggacttctagtataggagaagaggaatcctaatcctagtaattatatcttagtagatatgtgcttagtctcttgttaattttttgtcttgagaggactatatatactgataaagtgataatttattcagagaataaagaatgggctccatgctttgtctatgtattctcttcttgagggcaatttacgcctcttgagaagggtggcttcgttgttgggctcttgaaggcccacgcagtccaacggagcccactaagcctaattctgtt

SEQ ID NO: 20 - DNA, 600 bp, Zea mays
  source 1..600: mol_type = genomic DNA
ttacaatcgaagacgagctcgcatcgtggcaacaaattttggtgctccaaagctttggaaaggttatcctcaacattgttgggatccatggcggtctagaatcaaccatatgaaacactagcggctagggttcggaggagatcattagggaggagaaggagaggtcaagtctaagttgggctaggtgagaacgatcatgggtagatggatggcagtaccgggtaattttcatcaacttacgattaatagtgggtcttttattaaaaataagctgatataagcatcttttgagaagcttatagggttatcataatctcaagtactagattatataattttatcacataagttgcttcatacttagtttgtctatcactagcttatttacatagaatttagattatataataccaaaccctaaatgacctgaaccaaagatacccagattgcttcacatcatttgtgtggcactgtggctacatgaggacgtgggaatgggcgaatggcgcggaacgacccccgataggtttccgatattgccctccccacgcgcacgatttaaccgatccgcccccgtttctttctttttctccacgagaa

SEQ ID NO: 21 - DNA, 600 bp, Zea mays
  source 1..600: mol_type = genomic DNA
tattcaaattctactccgtaaatagtgcagcacacgacacaaaatagtgttttgggtaatgaaatgctggagacggtccgaatgtcgaccgaagcttttcagacatgctcgctccaaatttaggtcagctgtctctaatctgtgactgcgcgctacgggtacgtaaattttgtagtagcagaacgctaaatctctttcgaatcactgtttttattatgtcgagcaaaaaaaacatagcttcgtcgtgaattttgaattataaatgatcagcgagaatatttcagctgaattaaaatgtcctatacagacaagaaagccttaaatttcggatgctgggaacggtatccgccgctgatcatcctgaggagtaatatagcataataattaaaattattaagcttccaacgccgatgccaacggtcagaaagttcgaaatcattgtgctacatgaacctggtgtaccatcctgcccgtccatcactcttgatctgaatggaatgtccgcatgccgctctcggcccaccatacaccgagcccttcggtcgtcgccagttttgaatttcgaacatgttaaaagccccgcctgcgctgcccaccacc

SEQ ID NO: 22 - DNA, 600 bp, Zea mays
  source 1..600: mol_type = genomic DNA
cttaaaatatttcacattatccaaaggacttgtatgaaagtagaagtattcgaattcgatccatataaaagggttgagatctggtccgtatctgaatccggtattcagtatctaatctgtacctattatatatctgtatccgtaataaaaattgaatatttaagatgttgatatttatttaatatttatccaacacaatttgataatatccgtattaaatctaaatgcaaataaaaaataaatataaatataaacgagtaatacctattacatacctctccgcacgggcaactgtccaaaaattcgaaattcatatatcataccacgccacgccgcgatctcgatccaccaaccgcgctgtataccggcgtaccggcccgctgttcggtgtccgcgaatgccatttcagacccacaatttcgtttcgactgcacgcgcttggtccatacaccggatccatcactgcccgttggccgttgcacttcgccacacgaacgtccagatgtgactcggggtccaccaaaggcaccgtacacggtacaccggttatctctccttcgccatttcaaataggtttaaacatcccgctcaatagccgga